SEQUENCE LISTING (49 sequences)

SEQ ID NO: 1 - protein, 227 aa, ARTIFICIAL SEQUENCE
  Anti-Beta-Klotho VH Domain
EVQLLETGGGLIQPGGSLRLSCAASGFNVSRNYMSWVRQAPGKGLEWVSIIYSGGRTYYADSVKGRFTISRDNSKNMLYLQMNSLSAEDTAVYYCAKRNMGISATAPYDYWGQGTLVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 227 aa, ARTIFICIAL SEQUENCE
  Anti-Beta-Klotho VH Domain
EVQLVETGGGLIQPGGSLRLSCAASGFNVSRNYMSWVRQAPGKGLEWVSIIYSGGRTYYADSVKGRFTISRDNSKNMLYLQMNSLRAEDTAVYYCAKRNMGITAAAPYDYWGQGTLVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 227 aa, ARTIFICIAL SEQUENCE
  Anti-Beta-Klotho VH Domain
EVQLLESGGGLVQPGGSLRLSCAASGFNVSRNYMSWVRQAPGKGLEWVSIIYSGGRTYYADSVKGRFTISRDNSKNTLYLQMNSLRAEDTAVYYCAKRNMGITATAPYDYWGQGTLVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, ARTIFICIAL SEQUENCE
  Anti-Beta-Klotho VH Domain
QVQLVESGGGLVKPGGSLRLSCAASGFTFSSYAMSWVRQAPGKGLEWVSAISGGGDSTDYADSVKGRFTISRDNSKNTLYLQMNSLRAEDTAVYYCAKDYEILTGYYNPYYFDHWGQGTLVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 231 aa, ARTIFICIAL SEQUENCE
  Anti-Beta-Klotho VH Domain
EVQLVESGGGLVQPGGSLRLSCAASGFTFSSYAMNWVRQAPGKGLEWVSAISGGGDSTDYADSVKGRFTISRDNSKNTLYLQMNSLRAEDTAVYYCAKDHDIWTGYYNPYYFDNWGQGTLVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 229 aa, ARTIFICIAL SEQUENCE
  Anti-Beta-Klotho VH Domain
QVQLVESGGGLVKPGGSLRLSCAASGFTVNSNYMSWVRQAPGKGLEWVSVIYSGGSTYYADSVKGRFTISRDNSKNTLYLQMNSLRAEDTAVYYCARRARGVIINKPDAFDIWGQGTMVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 229 aa, ARTIFICIAL SEQUENCE
  Anti-Beta-Klotho VH Domain
QVQLVESGGGLVQPGGSLRLSCAASGFTFSDYYMSWVRQAPGKGLEWVSVIYSGGSTYYADSVKGRFTISRDNSKNTLYLQMNSLRAEDTAVYYCARRARGLIINKSDAFDIWGQGTMVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 226 aa, ARTIFICIAL SEQUENCE
  Anti-Beta-Klotho VH Domain
EVQLVESGGGLVKPGGSLRLSCAASGFTVSSYYMSWVRQAPGKGLEWVSIIYSGNNTYYADSVKGRFTISRDNSKNTLYLQMNSLRVEDTAVYYCARRGISVAGPIFDYWGQGTLVTVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 225 aa, ARTIFICIAL SEQUENCE
  Anti-Beta-Klotho VH Domain
EVQLVESGGGLAKPGGSLRLSCAASGFTFRNAWMSWVRQAPGKGLEWVGRIKSKTDGGTTDYAAPVKGRFTISRDDSKNTLYLQMNSLKTEDTAEYYCITDRVLSYYAMAVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - protein, 226 aa, ARTIFICIAL SEQUENCE
  Anti-Beta-Klotho VH Domain
QVQLVQSGAEVKKPGASVKVSCRASGYTFTSFDINWVRQATGQGLEWMGWMNPNSGNTDYAQKFQGRVTMTRNTSISTAYMELSDLRSEDTAVYFCARGGSWHYYFYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 11 - protein, 224 aa, ARTIFICIAL SEQUENCE
  Anti-Beta-Klotho VH Domain
EVQLLESGGGLVQPGGSLRLSCAASGFTFSIYAMSWVRQAPGKGLEWVSAISGSGGGTFYADSVKGRFTISRDNSKNTLFLQMNSLRAEDTAVYYCAKDRRIAVAGTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 12 - protein, 224 aa, ARTIFICIAL SEQUENCE
  Anti-Beta-Klotho VH Domain
EVQLLESGGGLVQPGGSLRLSCAASGFTFSSYAMSWVRQAPGKGLEWVSAISGSGAGTYYADSVKGRFTISRDNSKNTLYLQMNSLRAEDTAVYYCAKDRVIAVAAVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 13 - protein, 223 aa, ARTIFICIAL SEQUENCE
  Anti-Beta-Klotho VH Domain
QVQLVESGGGVVQPGRSLRLSCAASGFTFISYGMHWVRQAPGKGLEWVAVIWFDGSINNYADSVKGRFTISRDNSKNMLYLQMNSLRAEDTALYYCTRAGIVGASWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 222 aa, ARTIFICIAL SEQUENCE
  Anti-Beta-Klotho VH Domain
QVQLVESGGGVVQPGRSLRLSCAASGFTFSYYYIHWVRQAPGKGLEWVALIWYDGSNKDYADSVKGRFTISRDNSKNTLYLHVNSLRAEDTAVYYCAREGTTRRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 15 - DNA, 681 bp, ARTIFICIAL SEQUENCE
  Nucleotide Encoding of Anti-Beta-Klotho VH Domain
gaggtgcagctgttggagactggaggaggcctgatccagcctggggggtccctgagactctcctgtgcagcctctgggttcaacgtcagtcgcaactatatgagttgggtccgccaggctccagggaaggggctggagtgggtctcaattatttatagcggtggtagaacatactacgcagactccgtgaagggccgattcaccatctccagagacaattccaagaatatgctgtatcttcaaatgaacagcctgagtgccgaggacacggccgtttattactgtgcgaaaaggaatatgggtatatcagcaactgccccatatgactactggggccagggaaccctggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - DNA, 681 bp, ARTIFICIAL SEQUENCE
  Nucleotide Encoding of Anti-Beta-Klotho VH Domain
gaggtgcagctggtggagactggaggaggcctgatccagcctggggggtccctgagactctcctgtgcagcctctgggttcaacgtcagtcgcaactatatgagctgggtccgccaggctccagggaaggggctggagtgggtctcaattatttatagcggtggtagaacatactacgcagactccgtgaagggccgattcaccatctccagagacaattccaagaatatgctgtatcttcaaatgaacagcctgagagccgaggacacggccgtttattactgtgcgaaaaggaatatgggtataacagcagctgccccgtatgactactggggccagggaaccctggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 17 - DNA, 681 bp, ARTIFICIAL SEQUENCE
  Nucleotide Encoding of Anti-Beta-Klotho VH Domain
gaggtgcagctgttggagtctgggggaggcttggtacagcctggggggtccctgagactctcctgtgcagcctctgggttcaacgtcagtcgcaactatatgagctgggtccgccaggctccagggaaggggctggagtgggtttcaattatttatagcggtggtagaacatactacgcagactccgtgaagggccggttcaccatctccagagacaattccaagaacacgctgtatcttcaaatgaacagcctgagagccgaggacacggccgtttattactgtgcgaaaaggaatatgggtataacagcaactgccccgtatgactactggggccagggaaccctggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - DNA, 693 bp, ARTIFICIAL SEQUENCE
  Nucleotide Encoding of Anti-Beta-Klotho VH Domain
caggtgcagctggtggagtctgggggaggcttggtcaagcctggagggtccctgagactctcctgtgcagcctctggattcacctttagcagctatgccatgagctgggtccgccaggctccagggaaggggctggagtgggtctcagctattagtggtggtggtgatagcacagactacgcagactccgtgaagggccggttcaccatctccagagacaattccaagaacacgctgtatctgcaaatgaacagcctgagagccgaggacacggccgtatattactgtgcgaaagattacgagattttgactggttattataacccgtactactttgaccactggggccagggaaccctggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 19 - DNA, 693 bp, ARTIFICIAL SEQUENCE
  Nucleotide Encoding of Anti-Beta-Klotho VH Domain
gaggtgcagctggtggagtctgggggaggcttggtacagcctggggggtccctgagactctcctgtgcagcctctggattcacctttagcagctatgccatgaactgggtccgccaggctccagggaaggggctggagtgggtctcagctattagtggtggtggtgatagcacagactacgcagactccgtgaagggccggttcaccatctccagagacaattccaagaacacgctgtatctgcaaatgaacagcctgagagccgaggacacggccgtatattactgtgcgaaagatcacgatatttggactggttattataacccgtactactttgacaactggggccagggaaccctggtcactgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - DNA, 687 bp, ARTIFICIAL SEQUENCE
  Nucleotide Encoding of Anti-Beta-Klotho VH Domain
caggtgcagctggtggagtctgggggaggcttggtcaagcctggagggtccctgagactctcctgtgcagcctctgggttcaccgtcaatagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtatattactgtgcgagaagggctcggggagttattataaacaaacctgatgcttttgatatctggggccaagggacaatggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 21 - DNA, 687 bp, ARTIFICIAL SEQUENCE
  Nucleotide Encoding of Anti-Beta-Klotho VH Domain
caggtgcagctggtggagtctgggggaggcttggtacagcctggggggtccctgagactctcctgtgcagcctctggattcaccttcagtgactactacatgagctgggtccgccaggctccagggaaggggctggagtgggtctcagttatttatagcggtggtagcacatactacgcagactccgtgaagggccgattcaccatctccagagacaattccaagaacacgctgtatctgcaaatgaacagcctgagagccgaggacacggccgtgtattactgtgcgagaagggctcggggacttattataaacaaatctgatgcttttgatatctggggccaagggacaatggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 22 - DNA, 678 bp, ARTIFICIAL SEQUENCE
  Nucleotide Encoding of Anti-Beta-Klotho VH Domain
gaggtgcagctggtggagtctgggggaggcttggtcaagcctggggggtccctgagactctcctgtgcagcctctgggttcaccgtcagtagctactacatgagctgggtccgccaggctccagggaaggggctggagtgggtctcaattatttatagcggtaataacacatactacgcagactccgtgaagggccgattcaccatctccagagacaattccaagaacacgctgtatcttcaaatgaacagcctgagagtcgaggacacggccgtctattactgtgcgagaagaggtatatcagtggctggtcccatctttgactattggggccagggaaccctggtcaccgtctcccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 23 - DNA, 675 bp, ARTIFICIAL SEQUENCE
  Nucleotide Encoding of Anti-Beta-Klotho VH Domain
gaggtccagctggtggagagcggaggtggactcgccaagccgggtggttctctgaggctgagctgtgccgcctccggcttcacattcaggaacgcctggatgagctgggttaggcaagctccaggtaaaggcctcgaatgggtcggccgcatcaaaagcaagactgatggtggaaccacagactacgccgctcctgttaagggacgcttcacaattagtcgtgatgattccaagaataccctgtacctgcagatgaactctctgaagacagaagacacagcagagtattattgcattactgaccgtgtgctgtcctactacgccatggctgtgtggggccagggaaccactgttaccgtgagctctgctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 24 - DNA, 678 bp, ARTIFICIAL SEQUENCE
  Nucleotide Encoding of Anti-Beta-Klotho VH Domain
caggtccaactggttcagtctggcgccgaggtgaagaagcccggagccagcgtgaaagtttcctgccgggcctccgggtacacctttaccagtttcgatatcaactgggtgcgccaggccacaggacagggtttggaatggatgggttggatgaaccctaacagtggtaacactgattatgctcaaaaattccaaggccgcgttaccatgaccagaaacaccagtatttccaccgcctatatggagctcagtgacctccggtccgaggataccgctgtgtatttctgcgccagaggtgggagctggcattattatttttactacggtctcgacgtctggggccagggcactaccgtgactgtgtcttccgctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 25 - DNA, 672 bp, ARTIFICIAL SEQUENCE
  Nucleotide Encoding of Anti-Beta-Klotho VH Domain
gaggtgcagctcctggagagcggcggagggctggtccaacccggcggctctctgcggctgtcctgtgcggctagtggatttaccttctctatctacgctatgagctgggtccgtcaggcaccgggtaagggactcgaatgggtgtccgctatctctggcagcggcggtggcactttctacgccgacagcgttaagggtcgcttcaccatctctcgtgacaactccaagaataccctgttcctccagatgaattccctgcgcgccgaggacactgctgtttattactgcgcgaaggatcggcggatcgccgtcgctggcacattcgattactggggccagggtactctggtgaccgtgtccagtgctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 26 - DNA, 672 bp, ARTIFICIAL SEQUENCE
  Nucleotide Encoding of Anti-Beta-Klotho VH Domain
gaggtgcaactcctggagtccggtggaggcctggtgcagcccggaggatctctgagactgtcttgcgcggcctccggattcactttctcctcctacgctatgtcttgggtgcggcaggcccccggcaagggactcgagtgggtgtccgccatctccggctccggagccggcacctattacgcggacagcgtgaagggccgcttcaccatctcccgcgacaactctaagaacactctgtacctgcagatgaactctctgcgtgcagaggacaccgctgtctactactgcgctaaggatcgcgtgattgccgtcgccgctgtcttcgactactggggtcaggggacactcgtgaccgtgtccagcgctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 27 - DNA, 669 bp, ARTIFICIAL SEQUENCE
  Nucleotide Encoding of Anti-Beta-Klotho VH Domain
caggttcagctggtggagagtggaggtggcgtcgtccagccaggccgcagcctgcggctctcctgtgctgcttccggctttacctttatctcttacggcatgcactgggtgcgccaggcccccggcaaggggttggagtgggttgctgtgatctggtttgacggctccatcaacaactacgccgatagtgtgaagggacgcttcactatcagcagggacaacagcaagaatatgctgtacctgcagatgaattccctccgcgctgaagacaccgcgctgtactactgcacacgggctggtatcgtgggggcttcctggtttgacccatgggggcagggtactctggtgactgtgtccagcgctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttccccgctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 28 - DNA, 666 bp, ARTIFICIAL SEQUENCE
  Nucleotide Encoding of Anti-Beta-Klotho VH Domain
caggttcagctggtcgagagcggcggcggtgtcgtgcagcccggccgctccctccggctgtcttgtgcggcctctgggttcacatttagctactattacatccactgggtgagacaggctccaggtaaaggactcgagtgggtggctctgatctggtacgatgggagtaacaaagactacgcagacagtgttaaaggcagattcaccattagtcgcgataattccaagaataccctgtacttgcacgtcaacagcctgcgcgccgaggatactgctgtgtactattgcgctcgcgagggcactacaaggagaggattcgactactggggtcagggcaccctggtcacagtcagcagcgctagcaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 29 - DNA, 24 bp, ARTIFICIAL SEQUENCE
  PCR Primer
caccatggagtttgggctgagctg

SEQ ID NO: 30 - DNA, 23 bp, ARTIFICIAL SEQUENCE
  PCR Primer
caccatggactggacctggaggg

SEQ ID NO: 31 - DNA, 25 bp, ARTIFICIAL SEQUENCE
  PCR Primer
cattatgcacctccacgccgtccac

SEQ ID NO: 32 - protein, 106 aa, ARTIFICIAL SEQUENCE
  Human immunoglobulin light chain constant region sequence
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 33 - protein, 106 aa, ARTIFICIAL SEQUENCE
  Human immunoglobulin light chain constant region sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 106 aa, ARTIFICIAL SEQUENCE
  Human immunoglobulin light chain constant region sequence
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 35 - protein, 106 aa, ARTIFICIAL SEQUENCE
  Human immunoglobulin light chain constant region sequence
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 36 - protein, 330 aa, ARTIFICIAL SEQUENCE
  Human immunoglobulin heavy chain constant region sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 330 aa, ARTIFICIAL SEQUENCE [sequence omitted]

SEQ ID NO: 38 - protein, 330 aa, ARTIFICIAL SEQUENCE
  Human immunoglobulin heavy chain constant region sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 330 aa, ARTIFICIAL SEQUENCE
  Human immunoglobulin heavy chain constant region sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 326 aa, ARTIFICIAL SEQUENCE
  Human immunoglobulin heavy chain constant region sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - protein, 22 aa, ARTIFICIAL SEQUENCE
  Signal Peptide
MDMRVPAQLLGLLLLWLRGARC

SEQ ID NO: 42 - protein, 19 aa, ARTIFICIAL SEQUENCE
  Signal Peptide
MAWALLLLTLLTQGTGSWA

SEQ ID NO: 43 - protein, 19 aa, ARTIFICIAL SEQUENCE
  Signal Peptide
MTCSPLLLTLLIHCTGSWA

SEQ ID NO: 44 - protein, 20 aa, ARTIFICIAL SEQUENCE
  Signal Peptide
MEAPAQLLFLLLLWLPDTTG

SEQ ID NO: 45 - protein, 19 aa, ARTIFICIAL SEQUENCE
  Signal Peptide
MEWTWRVLFLVAAATGAHS

SEQ ID NO: 46 - protein, 20 aa, ARTIFICIAL SEQUENCE
  Signal Peptide
METPAQLLFLLLLWLPDTTG

SEQ ID NO: 47 - protein, 20 aa, ARTIFICIAL SEQUENCE
  Signal Peptide
METPAQLLFLLLLWLPDTTG

SEQ ID NO: 48 - protein, 19 aa, ARTIFICIAL SEQUENCE
  Signal Peptide
MKHLWFFLLLVAAPRWVLS

SEQ ID NO: 49 - protein, 19 aa, ARTIFICIAL SEQUENCE
  Signal Peptide
MEWSWVFLFFLSVTTGVHS

(19) United States
(12) Patent Application Publication
(10) Pub. No.: US 2023/0137351 A1
(43) Pub. Date: May 4, 2023

(54) BISPECIFIC OR BIPARATOPIC ANTIGEN BINDING PROTEINS AND USES THEREOF

(71) Applicant: AMGEN INC., THOUSAND OAKS, CA (US)

(72) Inventors: Wei YAN, Sammamish, WA (US); Zhi LIU, Shoreline, WA (US); Christopher MURAWSKY, Roberts Creek (CA); Chadwick Terence KING, North Vancouver (CA); Yang LI, Mountain View, CA (US); Zhonghua HU, Kenmore, WA (US); Desiree LIM, Coquitlam (CA)

(73) Assignee: AMGEN INC., THOUSAND OAKS, CA (US)

(21) Appl. No.: 16/349,965

(22) PCT Filed: Nov. 14, 2017
(86) PCT No.: PCT/US2017/061636

§ 371 Date: May 14, 2019

Related U.S. Application Data
(60) Provisional application No. 62/421,947, filed on Nov. 14, 2016.

(51) Int. Cl. C07K 16/46

(52) U.S. Cl. CPC C07K 16/468; C07K 2317/565; C07K 2317/51; C07K 2317/35

(57) ABSTRACT

The present invention relates to bispecific or biparatopic antigen binding proteins, polynucleotides encoding the same, and methods of making bispecific or biparatopic antigen binding proteins. Also described herein is a method to assemble IgG-like biparatopic or bispecific antibodies from VH only antigen binding proteins.

This application claims the benefit of U.S. Provisional Application No. 62/421,947, filed on Nov. 14, 2016, which is hereby incorporated by reference in its entirety.

The instant application contains a sequence listing which has been submitted electronically in ASCII format and is hereby incorporated by reference in its entirety. Said ASCII copy, created on Nov. 14, 2017, is named A-2110-WO-PCT_SeqListFinall11417_ST25.txt and is 65 kilobytes in size.

### FIELD OF THE INVENTION

The present invention relates to bispecific or biparatopic antigen binding proteins, polynucleotides encoding the same, and methods of making bispecific or biparatopic antigen binding proteins.

### BACKGROUND OF THE INVENTION

Mice can be engineered to produce antibodies with only heavy chain. Multiple studies have shown that some of these transgenic mice can mount a normal immune response and produce high affinity antibodies with only human heavy chains. This approach provides an opportunity to isolate minimum antigen specific binding unit with one Ig domain, which can be used as building blocks to assemble more complex molecules that can recognize more than one epitope. Described herein is a method to assemble IgG-like biparatopic or bispecific antibodies from VH only binders.

### SUMMARY OF THE INVENTION

The present invention is directed to a bispecific antigen binding protein, comprising:

a) a first polypeptide comprising a first heavy chain variable region (VH1), wherein the VH1 is fused through its C-terminus to the N-terminus of a CH1 domain and wherein the VH1 comprises three CDRs and binds to a first epitope, and

b) a second polypeptide comprising a second heavy chain variable region (VH2), wherein the VH2 is fused through its C-terminus to the N-terminus of a CL domain and wherein the VH2 comprises three CDRs and binds to a second epitope.

In certain embodiments the first and second epitopes are located on the same antigen. Alternatively, the first and second epitopes are located on different antigens.

### BRIEF DESCRIPTION OF THE DRAWINGS

FIG. 1 shows Harbour Mice—Transgenic mice which make fully human heavy chain only (HCO) antibodies.

FIG. 2 shows Identification of KLB VH Clones by yeast display.

FIG. 3 shows Confirming binding of selected VH yeast binders to the AMID cell expressing beta-Klotho/FGFR1c.

FIG. 4 shows Development of luciferase report assay o screen for beta-Klotho/FGFR1c agonists using VH displayed yeast.

FIG. 5 shows Screening of individual yeast clones for agonists in the luciferase reporter assay.

FIG. 6 shows Sequence alignment of 11 unique beta-Klotho/FGFR1c agonists.

FIG. 7 shows Biparatopic IgGs for KLB.

FIG. 8 shows VHO clones are more fit to build biparatopic IgG than VH from Xenomouse.

FIG. 9 shows VHO clones can also be paired with regular LC from Xenomouse to produce well-behaved IgG.

FIG. 10 shows Purification profiles of some Protein A-purified proteins.

FIG. 11 shows Biparatopic IgGs exhibit potent agonistic activity in luciferase reporter assay and adipocyte pERK assay.

FIG. 12 shows Modular assembly of various Fc based bispecific orbiparatopic fusions from VHO binders.

FIG. 13 shows One Harbor mice from 8V3 strain had immune response to soluble FGFR1c ECD.

FIG. 14 shows RT-PCR to clone VHO fragments for yeast display.

FIG. 15 shows Display α-KLB/FGFR1c VHOs on yeast surface.

FIG. 16 shows 20 FGFR1c specific VHO binders were identified.

FIG. 17 shows Assembly of bispecific Fab-Fc in library format.

### DETAILED DESCRIPTION OF THE INVENTION

The biparatopic or bispecific IgG has a similar configuration to naturally occurring IgG molecules which contain four polypeptide chains consisting of two identical heavy chains and two identical light chains. Each chain confers one antigen-specific binding unit, which we define as VH1 and VH2 respectively. VH1 and VH2 can be derived from heavy chain only transgenic mice and can bind the same or different epitopes of antigens. The heavy chain of the biparatopic or bispecific IgG contains the following domains from the N-terminal: VH1 of antigen binding domain, a CH1 domain, and an Fc domain. The light chain contains at least the following domains from the N-terminal: a VH2 domain, and a light chain constant domain (Cu or CX). Structurally the biparatopic or bispecific IgG is very similar to that of a conventional IgG, except the VL domain is replaced by VH2. Therefore it is expected to maintain all the drug-like properties of human IgG, such as good stability and pharmacokinetic profile in vivo.

In this configuration, the VH1 and VH2 are brought together by the close interaction between CH1 and CL domain. This allows the molecule to efficiently recognize the two distinct epitopes that are in close proximity. The bivalent nature of the design also allows efficient crosslinking of the two target molecules. This could be very useful in the design of receptor agonist using this format.

As used herein, the term “antigen binding protein” refers to a protein that specifically binds to one or more target antigens. Functional fragments of antigen binding proteins of the present invention include heavy chain variable regions (VH).

The VHs of the present invention may be derived from many sources, such as heavy chain antibodies (HCAb). Exceptions to the H2L2 structure of conventional antibodies also occur in some isotypes of the immunoglobulins found in camelids (camels, dromedaries and llamas; Hamers-Casterman et al., 1993 Nature 363: 446; Nguyen et al., 1998 J. Mol. Biol. 275: 413), wobbegong sharks (Nuttall et al., Mol. Immunol. 38:313-26, 2001), nurse sharks (Greenberg et al., Nature374:168-73, 1995; Roux et al., 1998 Proc. Nat. Acad. Sci. USA 95: 11804), and in the spotted ratfish (Nguyen, et al., “Heavy-chain antibodies in Camelidae; a case of evolutionary innovation,” 2002 Immunogenetics 54(1): 39-47). These antibodies can apparently form antigen-binding regions using only heavy chain variable region, in that these functional antibodies are dimers of heavy chains only (referred to as “heavy-chain antibodies” or “HCAbs”). Heavy chain antibodies that are a class of IgG and devoid of light chains are produced by animals of the genus Camelidae which includes camels, dromedaries and llamas (Hamers-Casterman et al., Nature 363:446-448 (1993)). Their binding domains consist only of the heavy-chain variable domains, often referred to as VHH to distinguish them from conventional VH. Muyldermans et al., J. Mol. Recognit. 12:131-140 (1999). The variable domain of the heavy-chain antibodies is sometimes referred to as a nanobody (Cortez-Retamozo et al., Cancer Research64:2853-57, 2004). A nanobody library may be generated from an immunized dromedary as described in Conrath et al., (Antimicrob Agents Chemother 45: 2807-12, 2001) or using recombinant methods.

Although the HCAbs are devoid of light chains, they have an antigen-binding repertoire. The genetic generation mechanism of HCAbs is reviewed in Nguyen et al. Adv. Immunol 79:261-296 (2001) and Nguyen et al., Immunogenetics 54:39-47 (2002). Sharks, including the nurse shark, display similar antigen receptor-containing single monomeric V-domains. Irving et al., J. Immunol. Methods 248:31-45 (2001); Roux et al., Proc. Natl. Acad. Sci. USA 95:11804 (1998).

VHHs comprise small intact antigen-binding fragments (for example, fragments that are about 15 kDa, 118-136 residues). Camelid VHH domains have been found to bind to antigen with high affinity (Desmyter et al., J. Biol. Chem. 276:26285-90, 2001), with VHH affinities typically in the nanomolar range and comparable with those of Fab and scFv fragments. VHHs are highly soluble and more stable than the corresponding derivatives of scFv and Fab fragments. VH fragments have been relatively difficult to produce in soluble form, but improvements in solubility and specific binding can be obtained when framework residues are altered to be more VHH-like. (See, for example, Reichman et al., J. Immunol Methods 1999, 231:25-38.).

Functional VHHs may be obtained by proteolytic cleavage of HCAb of an immunized camelid, by direct cloning of VHH genes from B-cells of an immunized camelid resulting in recombinant VHHs, or from naive or synthetic libraries. VHHs with desired antigen specificity may also be obtained through phage display methodology. Using VHHs in phage display is much simpler and more efficient compared to Fabs or scFvs, since only one domain needs to be cloned and expressed to obtain a functional antigen-binding fragment. Muyldermans, Biotechnol. 74:277-302 (2001); Ghahroudi et al., FEBS Lett. 414:521-526 (1997); and van der Linden et al., J. Biotechnol. 80:261-270 (2000). Methods for generating antibodies having camelid heavy chains are also described in U.S. Patent Publication Nos. 20050136049 and 20050037421.

Ribosome display methods may be used to identify and isolate VHH molecules having the desired binding activity and affinity. Irving et al., J. Immunol. Methods 248:31-45 (2001). Ribosome display and selection has the potential to generate and display large libraries (1014).

Other embodiments provide VHH-like molecules generated through the process of camelisation, by modifying non-Camelidae VHs, such as human VHHs, to improve their solubility and prevent non-specific binding. This is achieved by replacing residues on the VLs side of VHs with VHH-like residues, thereby mimicking the more soluble VHH fragments. Camelised VHfragments, particularly those based on the human framework, are expected to exhibit a greatly reduced immune response when administered in vivo to a patient and, accordingly, are expected to have significant advantages for therapeutic applications. Davies et al., FEBS Lett. 339:285-290 (1994); Davies et al., Protein Eng. 9:531-537 (1996); Tanha et al., J. Biol. Chem. 276:24774-24780 (2001); and Riechmann et al., Immunol. Methods 231:25-38 (1999).

VHs may also be produced by transgenic mice. The transgenic mouse (also referred to herein as HC transgenic mouse) is devoid of functional endogenous murine immunoglobulin loci (heavy chain, lambda light chain and kappa light chain). A HC transgenic mouse lacks the ability to produce endogenous murine immunoglobulins and will instead express heavy chain only antibodies comprising human VH domains, devoid of a light chain. For example, the mouse may express heavy chain only antibodies, comprising a human VH domain and an Fc domain derived from a non-human mammal. In a further example the mouse may express heavy chain only antibodies comprising a human VH domain and a human Fc domain. Alternatively the mouse may express heavy chain only antibodies comprising a human VH domain and a murine Fc domain. Heavy chain only antibodies may be obtained from HC transgenic mice expressing human VH and human Fc or human VH and murine Fc domains. Only B cells expressing heavy chain-only antibodies will be expanded in these mice. The generation of HC transgenic mice is undertaken by functionally silencing murine immunoglobulin loci. Specifically, methods used to silence the mouse heavy chain locus (WO2004/076618 & Ren, L, et al., Genomics 84 (2004), 686-695), the mouse lambda locus (WO03000737 & Zou, X., et al., EJI, 1995, 25, 2154-2162 and the kappa locus (Zou, X., et al., J1 2003 170, 1354-1361) have been described previously. Briefly, large scale deletions of the mouse heavy chain constant region and the mouse lambda chain locus result in silencing of these two immunoglobulin chains. The kappa light chain is silenced via a targeted insertion of a neomycin resistant cassette. Mice with dual silencing of the endogenous light chains (kappa and lambda) are created by conventional breeding (Zou, X., et al., JA 2003 170, 1354-1361). These light chain-KO mice are further bred with heavy chain KO mice to give triple heterozygous animals for breeding to derive a ‘triple knockout’ (TKO) line.

“Heavy” and “light” chains refer to the two polypeptides which comprise an IgG. A heavy chain can be broken down into the following domains from N-terminus to C-terminus: VH, CH1, CH2, and CH3. A light chain can be broken down into the following domains from N-terminus to C-terminus: VL and CL. The CH1 and CL domains will interact such that the VH and VL domains form a functional conformation.

As used herein, an antigen binding protein “specifically binds” to a target antigen when it has a significantly higher binding affinity for, and consequently is capable of distinguishing, that antigen, compared to its affinity for other unrelated proteins, under similar binding assay conditions. Antigen binding proteins that specifically bind an antigen may have an equilibrium dissociation constant (K_{D})≤1×10⁻⁶ M. The antigen binding protein specifically binds antigen with “high affinity” when the K_{D} is ≤1×10⁻⁸ M. In one embodiment, the antigen binding proteins of the invention bind to target antigen(s) with a K_{D} of ≤5×10⁻⁷ M. In another embodiment, the antigen binding proteins of the invention bind to target antigen(s) with a K_{D} of ≤1×10⁻⁷ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by an equilibrium/solution method. In certain embodiments, affinity is determined by a FACS binding assay. In certain embodiments of the invention, the antigen binding protein specifically binds to target antigen(s) expressed by a mammalian cell (e.g., CHO, HEK 293, Jurkat), with a K_{D} of 20 nM (2.0×10⁻⁸ M) or less, K_{D} of 10 nM (1.0×10⁻⁸ M) or less, K_{D} of 1 nM (1.0×10⁻⁹ M) or less, K_{D} of 500 pM (5.0×10⁻¹⁰ M) or less, K_{D} of 200 pM (2.0×10⁻¹⁰ M) or less, K_{D} of 150 pM (1.50×10⁻¹⁰ M) or less, K_{D} of 125 pM (1.25×10⁻¹⁰ M) or less, K_{D} of 105 pM (1.05×10⁻¹⁰ M) or less, K_{D} of 50 pM (5.0×10⁻¹¹ M) or less, or K_{D} of 20 pM (2.0×10⁻¹¹ M) or less, as determined by a Kinetic Exclusion Assay, conducted by the method described in Rathanaswami et al. Analytical Biochemistry, Vol. 373:52-60, 2008. In some embodiments, the antigen binding proteins described herein exhibit desirable characteristics such as binding avidity as measured by k_{d} (dissociation rate constant) for target antigen(s) of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for target antigen(s) of about 10⁻⁹, 10⁻¹⁰, 10⁻¹¹, 10⁻¹², 10⁻¹³, 10⁻¹⁴, 10⁻¹⁵, 10⁻¹⁶ M or lower (lower values indicating higher binding affinity).

In certain embodiments of the invention, the antigen binding proteins are bivalent or tetravalent. The valency of the binding protein denotes the number of individual antigen binding domains within the binding protein. For example, the terms “bivalent,” and “tetravalent” with reference to the antigen binding proteins of the invention refer to binding proteins with two and four antigen binding domains, respectively. Thus, a tetravalent antigen binding protein comprises four or more antigen binding domains. In other embodiments, the antigen binding proteins are bivalent. For instance, in certain embodiments, the tetravalent antigen binding proteins are tetraspecific comprising four antigen-binding domains: one to antigen-binding domain binding to a first target antigen, one antigen-binding domain binding to a second target antigen, one to antigen-binding domain binding to a third target antigen, and one antigen-binding domain binding to a fourth target antigen. Such molecules comprise four different VH domains and use bispecific antibody engineering technology to produce proper CH1/CL and CH3/CH3 interactions.

In one embodiment the bivalent bispecific antibody binds two distinct targets on two different cell types. An exemplary embodiment includes a bivalent bispecific antibody bridging between target tumor cell and a natural killer cell to direct the natural killer cell to the tumor. In yet another embodiment of the invention, the bivalent bispecific antibody binds two different epitopes on the same molecular target (i.e. biparatopic). It is also apparent to the one skilled in the art that one or both of the targets of the bivalent bispecific antibody can be soluble or expressed on a cell surface.

As used herein, the term “antigen binding domain,” which is used interchangeably with “binding domain,” refers to the region of the antigen binding protein that contains the amino acid residues that interact with the antigen and confer on the antigen binding protein its specificity and affinity for the antigen. In some embodiments, the binding domain may be derived from the natural ligands of the target antigen(s). As used herein, the term “target antigen(s)” refers to a first target antigen and/or a second target antigen of a bispecific molecule and also refers to a first target antigen, a second target antigen, a third target antigen, and/or a fourth target antigen of a tetraspecific molecule.

In certain embodiments of the antigen binding proteins of the invention, the VH domain may be derived from an antibody or functional fragment thereof. For instance, the VH domains of the antigen binding proteins of the invention may comprise one or more complementarity determining regions (CDR) from the heavy chain variable regions of antibodies that specifically bind to target antigen(s). As used herein, the term “CDR” refers to the complementarity determining region (also termed “minimal recognition units” or “hypervariable region”) within antibody variable sequences. There are three heavy chain variable region CDRs (CDRH1, CDRH2 and CDRH3). The term “CDR region” as used herein refers to a group of three CDRs that occur in a single variable region (i.e. the three three heavy chain CDRs). The CDRs typically are aligned by the framework regions to form a structure that binds specifically with a specific epitope or domain on the target protein. From N-terminus to C-terminus, naturally-occurring light and heavy chain variable regions both typically conform with the following order of these elements: FR1, CDR1, FR2, CDR2, FR3, CDR3 and FR4.

Both the EU index as in Kabat et al., Sequences of Proteins of Immunological Interest, 5th Ed. Public Health Service, National Institutes of Health, Bethesda, Md. (1991) and AHo numbering schemes (Honegger A. and Pluckthun A. J Mol Biol. 2001 Jun. 8; 309(3):657-70) can be used in the present invention. Amino acid positions and complementarity determining regions (CDRs) and framework regions (FR) of a given antibody may be identified using either system. For example, EU heavy chain positions of 39, 44, 183, 356, 357, 370, 392, 399, and 409 are equivalent to AHo heavy chain positions 46, 51, 230, 484, 485, 501, 528, 535, and 551, respectively. Similarly, EU light chain positions 38, 100, and 176 are equivalent to AHO light chain positions 46 141, and 230, respectively. Tables 1, 2, and 3 below demonstrate the equivalence between numbering positions.

TABLE 1
v1

| Chain | Domain | Mutation | AHo # | EU # | Kabat # |
|---|---|---|---|---|---|
| LC-E | Constant | E | 230 | 176 | 176 |
| LC-K | Constant | K | 230 | 176 | 176 |
| HC-E | CH1 | E | 230 | 183 | 188 |
| HC-K | CH1 | K | 230 | 183 | 188 |

TABLE 2
v2

| Chain | Domain | Mutation | AHo # | EU # | Kabat # |
|---|---|---|---|---|---|
| LC-E | Constant | E | 230 | 176 | 176 |
| LC-K | Constant | K | 230 | 176 | 176 |
| HC-E | Variable | E | 46 | 39 | 39 |
| | CH1 | E | 230 | 183 | 188 |
| HC-K | Variable | K | 46 | 39 | 39 |
| | CH1 | K | 230 | 183 | 188 |

TABLE 3
v3

| Chain | Domain | Mutation | AHo # | EU # | Kabat # |
|---|---|---|---|---|---|
| LC-E | Constant | E | 230 | 176 | 176 |
| LC-K | Constant | K | 230 | 176 | 176 |
| HC-E | Variable | E | 51 | 44 | 44 |
| | CH1 | E | 230 | 183 | 188 |
| HC-K | Variable | K | 51 | 44 | 44 |
| | CH1 | K | 230 | 183 | 188 |

The “heavy chain variable region,” used interchangeably herein with “VH domain” or “VH”, refers to the region in a heavy immunoglobulin chains which is involved directly in binding the antibody to the antigen. As discussed above, the regions of variable heavy chains have the same general structure and each region comprises four framework (FR) regions whose sequences are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site.

The “immunoglobulin domain” represents a peptide comprising an amino acid sequence similar to that of immunoglobulin and comprising approximately 100 amino acid residues including at least two cysteine residues. Examples of the immunoglobulin domain include VH, CH1, CH2, and CH3 of an immunoglobulin heavy chain, and VL and CL of an immunoglobulin light chain. In addition, the immunoglobulin domain is found in proteins other than immunoglobulin. Examples of the immunoglobulin domain in proteins other than immunoglobulin include an immunoglobulin domain included in a protein belonging to an immunoglobulin super family, such as a major histocompatibility complex (MHC), CD1, B7, T-cell receptor (TCR), and the like. Any of the immunoglobulin domains can be used as an immunoglobulin domain for the multivalent antibody of the present invention.

In a human antibody, CH1 means a region having the amino acid sequence at positions 118 to 215 of the EU index. A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a κ chain of a human antibody, CL represents a region having the amino acid sequence at positions 108 to 214 of the EU index. In a λ chain, CL represents a region having the amino acid sequence at positions 108 to 215.

The binding domains that specifically bind to target antigen(s) can be derived a) from known antibodies to these antigens or b) from new antibodies or antibody fragments obtained by de novo immunization methods using the antigen proteins or fragments thereof, by phage display, or other routine methods. The antibodies from which the binding domains for the bispecific and tetraspecific antigen binding proteins are derived can be monoclonal antibodies, polyclonal antibodies, recombinant antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with a target antigen(s) immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds target antigen(s).

Monoclonal antibodies secreted by a hybridoma cell line can be purified using any technique known in the art, such as protein A-Sepharose, hydroxylapatite chromatography, gel electrophoresis, dialysis, or affinity chromatography. Hybridomas or mAbs may be further screened to identify mAbs with particular properties, such as the ability to bind cells expressing target antigen(s), ability to block or interfere with the binding of target antigen(s) to their respective receptors or ligands, or the ability to functionally block either of target antigen(s).

In some embodiments, the binding domains of the bispecific and tetraspecific antigen binding proteins of the invention may be derived from humanized antibodies against target antigen(s). A “humanized antibody” refers to an antibody in which regions (e.g. framework regions) have been modified to comprise corresponding regions from a human immunoglobulin. Generally, a humanized antibody can be produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089 and 5,693,762; Jones et al., Nature, Vol. 321:522-525, 1986; Riechmann et al., Nature, Vol. 332:323-27, 1988; Verhoeyen et al., Science, Vol. 239:1534-1536, 1988). The CDRs of heavy chain variable regions of antibodies generated in another species can be grafted to consensus human FRs. To create consensus human FRs, FRs from several human heavy chain amino acid sequences may be aligned to identify a consensus amino acid sequence.

New antibodies generated against the target antigen(s) from which binding domains for the bispecific and tetraspecific antigen binding proteins of the invention can be derived can be fully human antibodies. A “fully human antibody” is an antibody that comprises variable and constant regions derived from human germ line immunoglobulin sequences. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,939,598; 5,545,807; 6,300,129; 6,255,458; 5,877,397; 5,874,299 and 5,545,806; in PCT publications WO91/10741, WO90/04036, WO 94/02602, WO 96/30498, WO 98/24893 and in EP 546073B1 and EP 546073A1.

The transgenic mice described above contain a human immunoglobulin gene minilocus that encodes unrearranged human heavy (mu and gamma) and kappa light chain immunoglobulin sequences, together with targeted mutations that inactivate the endogenous mu and kappa chain loci (Lonberg et al., 1994, Nature 368:856-859). Accordingly, the mice exhibit reduced expression of mouse IgM or kappa and in response to immunization, and the introduced human heavy and light chain transgenes undergo class switching and somatic mutation to generate high affinity human IgG kappa monoclonal antibodies (Lonberg et al., supra.; Lonberg and Huszar, 1995, Intern. Rev. Immunol. 13: 65-93; Harding and Lonberg, 1995, Ann. N.Y Acad. Sci. 764:536-546). The preparation of HuMab mice is described in detail in Taylor et al., 1992, Nucleic Acids Research 20:6287-6295; Chen et al., 1993, International Immunology 5:647-656; Tuaillon et al., 1994, J. Immunol. 152:2912-2920; Lonberg et al., 1994, Nature 368:856-859; Lonberg, 1994, Handbook of Exp. Pharmacology 113:49-101; Taylor et al., 1994, International Immunology 6:579-591; Lonberg and Huszar, 1995, Intern. Rev. Immunol. 13:65-93; Harding and Lonberg, 1995, Ann. N.Y Acad. Sci. 764:536-546; Fishwild et al., 1996, Nature Biotechnology 14:845-851; the foregoing references are hereby incorporated by reference in their entirety for all purposes. See, further U.S. Pat. Nos. 5,545,806; 5,569,825; 5,625,126; 5,633,425; 5,789,650; 5,877,397; 5,661,016; 5,814,318; 5,874,299; and 5,770,429; as well as U.S. Pat. No. 5,545,807; International Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., 1997, Nature Genetics 15:146-156, which are hereby incorporated by reference.

Human-derived antibodies can also be generated using phage display techniques. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into bispecific and tetraspecific antibody fragments with a second binding site capable of triggering an effector function, if desired. The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:
Algorithm: Needleman et al., 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

As used herein, the term “antibody” refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be kappa (κ) or lambda (λ). The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

The term “constant region” as used herein refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes. Examples of human immunoglobulin light chain constant region sequences are shown in the following table.

TABLE 4
Exemplary Human Immunoglobulin Light
Chain Constant Regions

| | SEQ | |
|---|---|---|
| | ID | CL Domain Amino |
| Designation | NO: | Acid Sequence |
| CL-1 | 32 | GQPKANPTVTLFPPSSEELQANKAT |
| | | LVCLISDFYPGAVTVAWKADGSPVK |
| | | AGVETTKPSKQSNNKYAASSYLSLT |
| | | PEQWKSHRSYSCQVTHEGSTVEKTV |
| | | APTECS |
| CL-2 | 33 | GQPKAAPSVTLFPPSSEELQANKAT |
| | | LVCLISDFYPGAVTVAWKADSSPVK |
| | | AGVETTTPSKQSNNKYAASSYLSLT |
| | | PEQWKSHRSYSCQVTHEGSTVEKTV |
| | | APTECS |
| CL-3 | 34 | GQPKAAPSVTLFPPSSEELQANKAT |
| | | LVCLISDFYPGAVTVAWKADSSPVK |
| | | AGVETTTPSKQSNNKYAASSYLSLT |
| | | PEQWKSHKSYSCQVTHEGSTVEKTV |
| | | APTECS |
| CL-7 | 35 | GQPKAAPSVTLFPPSSEELQANKAT |
| | | LVCLVSDFYPGAVTVAWKADGSPVK |
| | | VGVETTKPSKQSNNKYAASSYLSLT |
| | | PEQWKSHRSYSCRVTHEGSTVEKTV |
| | | APAECS |

The heavy chain constant region of the heterodimeric antibodies can be, for example, an alpha-, delta-, epsilon-, gamma-, or mu-type heavy chain constant region, e.g., a human alpha-, delta-, epsilon-, gamma-, or mu-type heavy chain constant region. In some embodiments, the heterodimeric antibodies comprise a heavy chain constant region from an IgG1, IgG2, IgG3, or IgG4 immunoglobulin. In one embodiment, the heterodimeric antibody comprises a heavy chain constant region from a human IgG1 immunoglobulin. In another embodiment, the heterodimeric antibody comprises a heavy chain constant region from a human IgG2 immunoglobulin. Examples of human IgG1 and IgG2 heavy chain constant region sequences are shown below in Table 5.

TABLE 5
Exemplary Human Immunoglobulin
Heavy Chain Constant Regions

| | SEQ | |
|---|---|---|
| Ig | ID | Heavy Chain Constant |
| isotype | NO: | Region Amino Acid Sequence |
| Human | 36 | ASTKGPSVFPLAPSSKSTSGGTAAL |
| IgG1z | | GCLVKDYFPEPVTVSWNSGALTSGV |
| | | HTFPAVLQSSGLYSLSSVVTVPSSS |
| | | LGTQTYICNVNHKPSNTKVDKKVEP |
| | | KSCDKTHTCPPCPAPELLGGPSVFL |
| | | FPPKPKDTLMISRTPEVTCVVVDVS |
| | | HEDPEVKFNWYVDGVEVHNAKTKPR |
| | | EEQYNSTYRVVSVLTVLHQDWLNGK |
| | | EYKCKVSNKALPAPIEKTISKAKGQ |
| | | PREPQVYTLPPSREEMTKNQVSLTC |
| | | LVKGFYPSDIAVEWESNGQPENNYK |
| | | TTPPVLDSDGSFFLYSKLTVDKSRW |
| | | QQGNVFSCSVMHEALHNHYTQKSLS |
| | | LSPGK |
| Human | 37 | ASTKGPSVFPLAPSSKSTSGGTAAL |
| IgG1za | | GCLVKDYFPEPVTVSWNSGALTSGV |
| | | HTFPAVLQSSGLYSLSSVVTVPSSS |
| | | LGTQTYICNVNHKPSNTKVDKKVEP |
| | | KSCDKTHTCPPCPAPELLGGPSVFL |
| | | FPPKPKDTLMISRTPEVTCVVVDVS |
| | | HEDPEVKFNWYVDGVEVHNAKTKPR |
| | | EEQYNSTYRVVSVLTVLHQDWLNGK |
| | | EYKCKVSNKALPAPIEKTISKAKGQ |
| | | PREPQVYTLPPSRDELTKNQVSLTC |
| | | LVKGFYPSDIAVEWESNGQPENNYK |
| | | TTPPVLDSDGSFFLYSKLTVDKSRW |
| | | QQGNVFSCSVMHEALHNHYTQKSLS |
| | | LSPGK |
| Human | 38 | ASTKGPSVFPLAPSSKSTSGGTAAL |
| IgG1f | | GCLVKDYFPEPVTVSWNSGALTSGV |
| | | HTFPAVLQSSGLYSLSSVVTVPSSS |
| | | LGTQTYICNVNHKPSNTKVDKRVEP |
| | | KSCDKTHTCPPCPAPELLGGPSVFL |
| | | FPPKPKDTLMISRTPEVTCVVVDVS |
| | | HEDPEVKFNWYVDGVEVHNAKTKPR |
| | | EEQYNSTYRVVSVLTVLHQDWLNGK |
| | | EYKCKVSNKALPAPIEKTISKAKGQ |
| | | PREPQVYTLPPSREEMTKNQVSLTC |
| | | LVKGFYPSDIAVEWESNGQPENNYK |
| | | TTPPVLDSDGSFFLYSKLTVDKSRW |
| | | QQGNVFSCSVMHEALHNHYTQKSLS |
| | | LSPGK |
| Human | 39 | ASTKGPSVFPLAPSSKSTSGGTAAL |
| IgG1fa | | GCLVKDYFPEPVTVSWNSGALTSGV |
| | | HTFPAVLQSSGLYSLSSVVTVPSSS |
| | | LGTQTYICNVNHKPSNTKVDKRVEP |
| | | KSCDKTHTCPPCPAPELLGGPSVFL |
| | | FPPKPKDTLMISRTPEVTCVVVDVS |
| | | HEDPEVKFNWYVDGVEVHNAKTKPR |
| | | EEQYNSTYRVVSVLTVLHQDWLNGK |
| | | EYKCKVSNKALPAPIEKTISKAKGQ |
| | | PREPQVYTLPPSRDELTKNQVSLTC |
| | | LVKGFYPSDIAVEWESNGQPENNYK |
| | | TTPPVLDSDGSFFLYSKLTVDKSRW |
| | | QQGNVFSCSVMHEALHNHYTQKSLS |
| | | LSPGK |
| Human | 40 | ASTKGPSVFPLAPCSRSTSESTAAL |
| IgG2 | | GCLVKDYFPEPVTVSWNSGALTSGV |
| | | HTFPAVLQSSGLYSLSSVVTVPSSN |
| | | FGTQTYTCNVDHKPSNTKVDKTVER |
| | | KCCVECPPCPAPPVAGPSVFLFPPK |
| | | PKDTLMISRTPEVTCVVVDVSHEDP |
| | | EVQFNWYVDGVEVHNAKTKPREEQF |
| | | NSTFRVVSVLTVVHQDWLNGKEYKC |
| | | KVSNKGLPAPIEKTISKTKGQPREP |
| | | QVYTLPPSREEMTKNQVSLTCLVKG |
| | | FYPSDIAVEWESNGQPENNYKTTPP |
| | | MLDSDGSFFLYSKLTVDKSRWQQGN |
| | | VFSCSVMHEALHNHYTQKSLSLSPG |
| | | K |

A VH region may be attached to the above heavy and light chain constant regions to form complete antibody heavy chains and VH/CL chains, respectively. Further, each of the so generated heavy chain and VH/CL polypeptides may be combined to form a complete bispecific and tetraspecific antibody structure. It should be understood that the heavy chain variable regions provided herein can also be attached to other constant domains having different sequences than the exemplary sequences listed above.

In certain embodiments of the invention two different heavy chains are used to form a heterodimeric molecule of the present invention. To facilitate assembly of the and VH/CL polypeptides and heavy chains from into a bispecific and tetraspecific, heterodimeric antibody, the VH/CL polypeptides and/or heavy chains from each antibody can be engineered to reduce the formation of mispaired molecules. For example, one approach to promote heterodimer formation over homodimer formation is the so-called “knobs-into-holes” method, which involves introducing mutations into the CH3 domains of two different antibody heavy chains at the contact interface. Specifically, one or more bulky amino acids in one heavy chain are replaced with amino acids having short side chains (e.g. alanine or threonine) to create a “hole,” whereas one or more amino acids with large side chains (e.g. tyrosine or tryptophan) are introduced into the other heavy chain to create a “knob.” When the modified heavy chains are co-expressed, a greater percentage of heterodimers (knob-hole) are formed as compared to homodimers (hole-hole or knob-knob). The “knobs-into-holes” methodology is described in detail in WO 96/027011; Ridgway et al., Protein Eng., Vol. 9: 617-621, 1996; and Merchant et al., Nat, Biotechnol., Vol. 16: 677-681, 1998, all of which are hereby incorporated by reference in their entireties.

Another approach for promoting heterodimer formation to the exclusion of homodimer formation entails utilizing an electrostatic steering mechanism (see Gunasekaran et al., J. Biol. Chem., Vol. 285: 19637-19646, 2010, which is hereby incorporated by reference in its entirety). This approach involves introducing or exploiting charged residues in the CH3 domain in each heavy chain so that the two different heavy chains associate through opposite charges that cause electrostatic attraction. Homodimerization of the identical heavy chains are disfavored because the identical heavy chains have the same charge and therefore are repelled. This same electrostatic steering technique can be used to prevent mispairing of VH/CL polypeptides with the non-cognate heavy chains by introducing residues having opposite charges in the correct VH/CL polypeptide—heavy chain pair at the binding interface. The electrostatic steering technique and suitable charge pair mutations for promoting heterodimers and correct VH/CL chain/heavy chain pairing is described in WO2009089004 and WO2014081955, both of which are hereby incorporated by reference in their entireties.

In embodiments in which the antigen binding proteins of the invention are heterodimeric antibodies comprising a first and VH/CL polypeptide (and VH/CL1) that specifically binds to a first target antigen; a first heavy chain (HC1) that specifically binds to a second target antigen; a second VH/CL polypeptide (VH/CL2) that specifically binds to a third target antigen; and a second heavy chain (HC2) that specifically binds to a fourth antigen, HC1 or HC2 may comprise one or more amino acid substitutions to replace a positively-charged amino acid with a negatively-charged amino acid. For instance, in one embodiment, the CH3 domain of HC1 or the CH3 domain of HC2 comprises an amino acid sequence differing from a wild-type IgG amino acid sequence such that one or more positively-charged amino acids (e.g., lysine, histidine and arginine) in the wild-type human IgG amino acid sequence are replaced with one or more negatively-charged amino acids (e.g., aspartic acid and glutamic acid) at the corresponding position(s) in the CH3 domain. In these and other embodiments, amino acids (e.g. lysine) at one or more positions selected from 370, 392 and 409 (EU numbering system) are replaced with a negatively-charged amino acid (e.g., aspartic acid and glutamic acid). An amino acid substitution in an amino acid sequence is typically designated herein with a one-letter abbreviation for the amino acid residue in a particular position, followed by the numerical amino acid position relative to an original sequence of interest, which is then followed by the one-letter symbol for the amino acid residue substituted in. For example, “T30D” symbolizes a substitution of a threonine residue by an aspartate residue at amino acid position 30, relative to the original sequence of interest. Another example, “S218G” symbolizes a substitution of a serine residue by a glycine residue at amino acid position 218, relative to the original amino acid sequence of interest.

In certain embodiments, HC1 or HC2 of the heterodimeric antibodies may comprise one or more amino acid substitutions to replace a negatively-charged amino acid with a positively-charged amino acid. For instance, in one embodiment, the CH3 domain of HC1 or the CH3 domain of HC2 comprises an amino acid sequence differing from wild-type IgG amino acid sequence such that one or more negatively-charged amino acids in the wild-type human IgG amino acid sequence are replaced with one or more positively-charged amino acids at the corresponding position(s) in the CH3 domain. In these and other embodiments, amino acids (e.g., aspartic acid or glutamic acid) at one or more positions selected from 356, 357, and 399 (EU numbering system) of the CH3 domain are replaced with a positively-charged amino acid (e.g., lysine, histidine and arginine).

In particular embodiments, the tetraspecfic antibody comprises a first heavy chain comprising negatively-charged amino acids at positions 392 and 409 (e.g., K392D and K409D substitutions), and a second heavy chain comprising positively-charged amino acids at positions 356 and 399 (e.g., E356K and D399K substitutions). In other particular embodiments, the heterodimeric antibody comprises a first heavy chain comprising negatively-charged amino acids at positions 392, 409, and 370 (e.g., K392D, K409D, and K370D substitutions), and a second heavy chain comprising positively-charged amino acids at positions 356, 399, and 357 (e.g., E356K, D399K, and E357K substitutions).

To facilitate the association of a particular heavy chain with its cognate VH/CL chain, both the heavy and VH/CL polypeptides may contain complimentary amino acid substitutions. As used herein, “complimentary amino acid substitutions” refer to a substitution to a positively-charged amino acid in one chain paired with a negatively-charged amino acid substitution in the other chain. For example, in some embodiments, the heavy chain comprises at least one amino acid substitution to introduce a charged amino acid and the corresponding VH/CL polypeptide comprises at least one amino acid substitution to introduce a charged amino acid, wherein the charged amino acid introduced into the heavy chain has the opposite charge of the amino acid introduced into the VH/CL chain. In certain embodiments, one or more positively-charged residues (e.g., lysine, histidine or arginine) can be introduced into a first VH/CL polypeptide (LC1) and one or more negatively-charged residues (e.g., aspartic acid or glutamic acid) can be introduced into the companion heavy chain (HC1) at the binding interface of CL/CH1, whereas one or more negatively-charged residues (e.g., aspartic acid or glutamic acid) can be introduced into a second VH/CL polypeptide and one or more positively-charged residues (e.g., lysine, histidine or arginine) can be introduced into the companion heavy chain (HC2) at the binding interface of that pair's CL/CH1 interface. The electrostatic interactions will direct the CL1 to pair with CH1-1 and CL2 to pair with CH1-2, as the opposite charged residues (polarity) at the interface attract. The heavy/VH/CL polypeptide pairs having the same charged residues (polarity) at an interface will repel, resulting in suppression of the unwanted CH1/CL pairings.

In these and other embodiments, the CH1 domain of the heavy chain or the CL domain of the VH/CL polypeptide comprises an amino acid sequence differing from wild-type IgG amino acid sequence such that one or more positively-charged amino acids in wild-type IgG amino acid sequence is replaced with one or more negatively-charged amino acids. Alternatively, the CH1 domain of the heavy chain or the CL domain of the VH/CL polypeptide comprises an amino acid sequence differing from wild-type IgG amino acid sequence such that one or more negatively-charged amino acids in wild-type IgG amino acid sequence is replaced with one or more positively-charged amino acids. In some embodiments, one or more amino acids in the CH1 domain of the first and/or second heavy chain in the heterodimeric antibody at an EU position selected from F126, P127, L128, A141, L145, K147, D148, H168, F170, P171, V173, Q175, S176, S183, V185 and K213 is replaced with a charged amino acid. In certain embodiments, a heavy chain residue for substitution with a negatively- or positively-charged amino acid is S183 (EU numbering system). In some embodiments, S183 is substituted with a positively-charged amino acid. In alternative embodiments, S183 is substituted with a negatively-charged amino acid. For instance, in one embodiment, S183 is substituted with a negatively-charged amino acid (e.g. S183E) in the first heavy chain, and S183 is substituted with a positively-charged amino acid (e.g. S183K) in the second heavy chain.

In embodiments in which the VH/CL polypeptide comprises a kappa light chain constant domain, one or more amino acids in the CL domain in the antigen binding protein at a position (EU numbering in a kappa light chain) selected from F116, F118, S121, D122, E123, Q124, S131, V133, L135, N137, N138, Q160, S162, T164, S174 and S176 is replaced with a charged amino acid. In embodiments in which the VH/CL polypeptide comprises a lambda light chain constant domain, one or more amino acids in the CL domain at a position (EU numbering in a lambda chain) selected from T116, F118, S121, E123, E124, K129, T131, V133, L135, S137, E160, T162, S165, Q167, A174, S176 and Y178 is replaced with a charged amino acid. In some embodiments, a residue for substitution with a negatively- or positively-charged amino acid is S176 (EU numbering system) of the CL domain of either a kappa or lambda VH/CL chain. In certain embodiments, S176 of the CL domain is replaced with a positively-charged amino acid. In alternative embodiments, S176 of the CL domain is replaced with a negatively-charged amino acid. In one embodiment, S176 is substituted with a positively-charged amino acid (e.g. S176K) in the first VH/CL chain, and S176 is substituted with a negatively-charged amino acid (e.g. S176E) in the second VH/CL chain.

In one embodiment the invention also includes antigen binding proteins comprising the heavy chain(s) and/or VH/CL chain(s), where one, two, three, four or five amino acid residues are lacking from the N-terminus or C-terminus, or both, in relation to any one of the heavy and VH/CL chains, e.g., due to post-translational modifications resulting from the type of host cell in which the antibodies are expressed. For instance, Chinese Hamster Ovary (CHO) cells frequently cleave off a C-terminal lysine from antibody heavy chains.

The heavy chain constant regions or the Fc regions of the antigen binding proteins described herein may comprise one or more amino acid substitutions that affect the glycosylation and/or effector function of the antigen binding protein. One of the functions of the Fc region of an immunoglobulin is to communicate to the immune system when the immunoglobulin binds its target. This is commonly referred to as “effector function.” Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc region to Fc receptors on the surface of cells of the immune system. CDC is mediated through the binding of the Fc with proteins of the complement system, e.g., C1q. In some embodiments, the antigen binding proteins of the invention comprise one or more amino acid substitutions in the constant region to enhance effector function, including ADCC activity, CDC activity, ADCP activity, and/or the clearance or half-life of the antigen binding protein. Exemplary amino acid substitutions (EU numbering) that can enhance effector function include, but are not limited to, E233L, L234L, L234Y, L235S, G236A, S239D, F243L, F243V, P2471, D280H, K290S, K290E, K290N, K290Y, R292P, E294L, Y296W, S298A, S298D, S298V, S298G, S298T, T299A, Y300L, V305I, Q31IM, K326A, K326E, K326W, A330S, A330L, A330M, A330F, I332E, D333A, E333S, E333A, K334A, K334V, A339D, A339Q, P396L, or combinations of any of the foregoing.

In other embodiments, the antigen binding proteins of the invention comprise one or more amino acid substitutions in the constant region to reduce effector function. Exemplary amino acid substitutions (EU numbering) that can reduce effector function include, but are not limited to, C220S, C226S, C229S, E233P, L234A, L234V, V234A, L234F, L235A, L235E, G237A, P238S, S267E, H268Q, N297A, N297G, V309L, E318A, L328F, A330S, A331S, P331S or combinations of any of the foregoing.

Glycosylation can contribute to the effector function of antibodies, particularly IgG1 antibodies. Thus, in some embodiments, the antigen binding proteins of the invention may comprise one or more amino acid substitutions that affect the level or type of glycosylation of the binding proteins. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In certain embodiments, glycosylation of the antigen binding proteins described herein is increased by adding one or more glycosylation sites, e.g., to the Fc region of the binding protein. Addition of glycosylation sites to the antigen binding protein can be conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence may be altered through changes at the DNA level, particularly by mutating the DNA encoding the target polypeptide at preselected bases such that codons are generated that will translate into the desired amino acids.

The invention also encompasses production of antigen binding protein molecules with altered carbohydrate structure resulting in altered effector activity, including antigen binding proteins with absent or reduced fucosylation that exhibit improved ADCC activity. Various methods are known in the art to reduce or eliminate fucosylation. For example, ADCC effector activity is mediated by binding of the antibody molecule to the FcγRIII receptor, which has been shown to be dependent on the carbohydrate structure of the N-linked glycosylation at the N297 residue of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (see Yamane-Ohnuki et al., Biotechnol Bioeng. 87(5):614-22, 2004). Similar effects can be accomplished through decreasing the activity of alpha-1,6-fucosyl transferase enzyme or other enzymes in the fucosylation pathway, e.g., through siRNA or antisense RNA treatment, engineering cell lines to knockout the enzyme(s), or culturing with selective glycosylation inhibitors (see Rothman et al., Mol Immunol. 26(12):1113-23, 1989). Some host cell strains, e.g. Lec13 or rat hybridoma YB2/0 cell line naturally produce antibodies with lower fucosylation levels (see Shields et al., J Biol Chem. 277(30):26733-40, 2002 and Shinkawa et al., J Biol Chem. 278(5):3466-73, 2003). An increase in the level of bisected carbohydrate, e.g. through recombinantly producing antibody in cells that overexpress GnTIII enzyme, has also been determined to increase ADCC activity (see Umana et al., Nat Biotechnol. 17(2):176-80, 1999).

In other embodiments, glycosylation of the antigen binding proteins described herein is decreased or eliminated by removing one or more glycosylation sites, e.g., from the Fc region of the binding protein. Amino acid substitutions that eliminate or alter N-linked glycosylation sites can reduce or eliminate N-linked glycosylation of the antigen binding protein. In certain embodiments, the antigen binding proteins described herein comprise a mutation at position N297 (EU numbering), such as N297Q, N297A, or N297G. In one particular embodiment, the antigen binding proteins of the invention comprise a Fc region from a human IgG1 antibody with a N297G mutation. To improve the stability of molecules comprising a N297 mutation, the Fc region of the molecules may be further engineered. For instance, in some embodiments, one or more amino acids in the Fc region are substituted with cysteine to promote disulfide bond formation in the dimeric state. Residues corresponding to V259, A287, R292, V302, L306, V323, or 1332 (EU numbering) of an IgG1 Fc region may thus be substituted with cysteine. In one embodiment, specific pairs of residues are substituted with cysteine such that they preferentially form a disulfide bond with each other, thus limiting or preventing disulfide bond scrambling. In certain embodiments pairs include, but are not limited to, A287C and L306C, V259C and L306C, R292C and V302C, and V323C and I332C. In particular embodiments, the antigen binding proteins described herein comprise a Fc region from a human IgG1 antibody with mutations at R292C and V302C. In such embodiments, the Fc region may also comprise a N297G mutation.

Modifications of the antigen binding proteins of the invention to increase serum half-life also may desirable, for example, by incorporation of or addition of a salvage receptor binding epitope (e.g., by mutation of the appropriate region or by incorporating the epitope into a peptide tag that is then fused to the antigen binding protein at either end or in the middle, e.g., by DNA or peptide synthesis; see, e.g., WO96/32478) or adding molecules such as PEG or other water soluble polymers, including polysaccharide polymers. The salvage receptor binding epitope preferably constitutes a region wherein any one or more amino acid residues from one or two loops of a Fc region are transferred to an analogous position in the antigen binding protein. In one embodiment, three or more residues from one or two loops of the Fc region are transferred. In one embodiment, the epitope is taken from the CH2 domain of the Fc region (e.g., an IgG Fc region) and transferred to the CH1, CH3, or VH region, or more than one such region, of the antigen binding protein. Alternatively, the epitope is taken from the CH2 domain of the Fc region and transferred to the CL region or VL region, or both, of the antigen binding protein. See International applications WO 97/34631 and WO 96/32478 for a description of Fc variants and their interaction with the salvage receptor.

The present invention includes one or more isolated nucleic acids encoding the antigen binding proteins and components thereof described herein. Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. In one embodiment, the nucleic acids of the invention are derived from human sources, but the invention includes those derived from non-human species, as well.

Relevant amino acid sequences from an immunoglobulin or region thereof (e.g. variable region, Fc region, etc.) or polypeptide of interest may be determined by direct protein sequencing, and suitable encoding nucleotide sequences can be designed according to a universal codon table. Alternatively, genomic or cDNA encoding monoclonal antibodies from which the binding domains of the antigen binding proteins of the invention may be derived can be isolated and sequenced from cells producing such antibodies using conventional procedures (e.g., by using oligonucleotide probes that are capable of binding specifically to genes encoding the heavy chains of the monoclonal antibodies).

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one embodiment, the nucleic acids are substantially free from contaminating endogenous material. The nucleic acid molecule has been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ production of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The present invention also includes nucleic acids that hybridize under moderately stringent conditions, and highly stringent conditions, to nucleic acids encoding polypeptides as described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tm) of the hybrid, where Tm is determined according to the following equations. For hybrids less than 18 base pairs in length, Tm (° C.)=2(# of A+T bases)+4(# of G+C bases). For hybrids above 18 base pairs in length, Tm (° C.)=81.5+16.6(log 10 [Na⁺])+0.41(% G+C)−(600/N), where N is the number of bases in the hybrid, and [Na⁺] is the concentration of sodium ions in the hybridization buffer ([Na⁺] for 1×SSC=0.165M). In one embodiment, each such hybridizing nucleic acid has a length that is at least 15 nucleotides (or at least 18 nucleotides, or at least 20 nucleotides, or at least 25 nucleotides, or at least 30 nucleotides, or at least 40 nucleotides, or at least 50 nucleotides), or at least 25% (or at least 50%, or at least 60%, or at least 70%, or at least 80%) of the length of the nucleic acid of the present invention to which it hybridizes, and has at least 60% sequence identity (or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

Variants of the antigen binding proteins described herein can be prepared by site-specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein. However, antigen binding proteins comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antigen binding proteins. Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In certain embodiments, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions and/or framework regions is contemplated. Covariance analysis techniques can be employed by the skilled artisan to design useful modifications in the amino acid sequence of the antigen binding protein. See, e.g., Choulier, et al., Proteins 41:475-484, 2000; Demarest et al., J. Mol. Biol. 335:41-48, 2004; Hugo et al., Protein Engineering 16(5):381-86, 2003; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antigen binding protein.

The nucleic acid sequences of the present invention. As will be appreciated by those in the art, due to the degeneracy of the genetic code, an extremely large number of nucleic acids may be made, all of which encode the CDRs (and heavy and light chains or other components of the antigen binding proteins described herein) of the invention. Thus, having identified a particular amino acid sequence, those skilled in the art could make any number of different nucleic acids, by simply modifying the sequence of one or more codons in a way which does not change the amino acid sequence of the encoded protein.

The present invention also includes vectors comprising one or more nucleic acids encoding one or more components of the antigen binding proteins of the invention (e.g. variable regions, VH/CL chains, heavy chains). The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the polypeptides sequences of the present invention. In certain embodiments, a signal peptide having the amino acid sequence of MDMRVPAQLLGLLLLWLRGARC (SEQ ID NO: 41) is fused to the amino terminus of any of the polypeptide sequences of the present invention. In other embodiments, a signal peptide having the amino acid sequence of MAWALLLLTLLTQGTGSWA (SEQ ID NO: 42) is fused to the amino terminus of any of the polypeptide sequences of the present invention. In still other embodiments, a signal peptide having the amino acid sequence of MTCSPLLLTLLIHCTGSWA (SEQ ID NO: 43) is fused to the amino terminus of any of the polypeptide sequences of the present invention. Other suitable signal peptide sequences that can be fused to the amino terminus of the polypeptide sequences described herein include: MEAPAQLLFLLLLWLPDTTG (SEQ ID NO: 44), MEWTWRVLFLVAAATGAHS (SEQ ID NO: 45), METPAQLLFLLLLWLPDTTG (SEQ ID NO: 46), METPAQLLFLLLLWLPDTTG (SEQ ID NO: 47), MKHLWFFLLLVAAPRWVLS (SEQ ID NO: 48), and MEWSWVFLFFLSVTTGVHS (SEQ ID NO: 49). Other signal peptides are known to those of skill in the art and may be fused to any of the polypeptide chains of the present invention, for example, to facilitate or optimize expression in particular host cells.

Typically, expression vectors used in the host cells to produce the bispecific antigen proteins of the invention will contain sequences for plasmid maintenance and for cloning and expression of exogenous nucleotide sequences encoding the components of the antigen binding proteins. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide tag sequence encodes polyHis (such as hexaHis), FLAG, HA (hemaglutinin influenza virus), myc, or another “tag” molecule for which commercially available antibodies exist. This tag is typically fused to the polypeptide upon expression of the polypeptide, and can serve as a means for affinity purification or detection of the polypeptide from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using routine methods for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using known methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as one or more components of the antigen binding proteins described herein. As a result, increased quantities of a polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning vectors of the invention will typically contain a promoter that is recognized by the host organism and operably linked to the molecule encoding the polypeptide. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system.

Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding e.g., heavy chain, VH/CL chain, modified heavy chain, or other component of the antigen binding proteins of the invention, by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315: 115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7: 1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5: 1639-1648; Hammer et al., 1987, Science 253:53-58); the alpha 1-antitrypsin gene control region that is active in liver (Kelsey et al., 1987, Genes and Devel. 1: 161-171); the beta-globin gene control region that is active in myeloid cells (Mogram et al, 1985, Nature 315:338-340; Kollias et al, 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234: 1372-1378).

An enhancer sequence may be inserted into the vector to increase transcription of DNA encoding a component of the antigen binding proteins (e.g., VH/CL chain, heavy chain, modified heavy chain) by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides are described above. Other signal peptides that are functional in mammalian host cells include the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

The expression vectors that are provided may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art. The expression vectors can be introduced into host cells to thereby produce proteins, including fusion proteins, encoded by nucleic acids as described herein.

After the vector has been constructed and the one or more nucleic acid molecules encoding the components of the antigen binding proteins described herein has been inserted into the proper site(s) of the vector or vectors, the completed vector(s) may be inserted into a suitable host cell for amplification and/or polypeptide expression. Thus, the present invention encompasses an isolated host cell comprising one or more expression vectors encoding the components of the antigen binding proteins. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated nucleic acid of the invention, in one embodiment operably linked to at least one expression control sequence (e.g. promoter or enhancer), is a “recombinant host cell.”

The transformation of an expression vector for an antigen binding protein into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

A host cell, when cultured under appropriate conditions, synthesizes an antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. In certain embodiments, cell lines may be selected through determining which cell lines have high expression levels and constitutively produce antigen binding proteins of the present invention. In another embodiment, a cell line from the B cell lineage that does not make its own antibody but has a capacity to make and secrete a heterologous antibody can be selected. CHO cells are host cells in some embodiments for expressing the antigen binding proteins of the invention.

Host cells are transformed or transfected with the above-described nucleic acids or vectors for production of antigen binding proteins and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antigen binding proteins. Thus, the present invention also provides a method for preparing a bispecific antigen binding protein described herein comprising culturing a host cell comprising one or more expression vectors described herein in a culture medium under conditions permitting expression of the bispecific antigen binding protein encoded by the one or more expression vectors; and recovering the bispecific antigen binding protein from the culture medium.

The host cells used to produce the antigen binding proteins of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO90103430; WO 87/00195; or U.S. Patent Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the bispecific antigen binding protein can be produced intracellularly, in the periplasmic space, or directly secreted into the medium. If the antigen binding protein is produced intracellularly, as a first step, the particulate debris, either host cells or lysed fragments, is removed, for example, by centrifugation or ultrafiltration. The bispecifc antigen binding protein can be purified using, for example, hydroxyapatite chromatography, cation or anion exchange chromatography, or affinity chromatography, using the antigen(s) of interest or protein A or protein G as an affinity ligand. Protein A can be used to purify proteins that include polypeptides that are based on human 71, 72, or 74 heavy chains (Lindmark et al., J. Immunol. Meth. 62: 1-13, 1983). Protein G is recommended for all mouse isotypes and for human 73 (Guss et al., EMBO J. 5: 15671575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a CH3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular bispecific antigen binding protein to be recovered.

In some embodiments, the invention provides a pharmaceutical composition comprising one or a plurality of the antigen binding proteins of the invention together with pharmaceutically acceptable diluents, carriers, excipients, solubilizers, emulsifiers, preservatives, and/or adjuvants. Pharmaceutical compositions of the invention include, but are not limited to, liquid, frozen, and lyophilized compositions. “Pharmaceutically-acceptable” refers to molecules, compounds, and compositions that are non-toxic to human recipients at the dosages and concentrations employed and/or do not produce allergic or adverse reactions when administered to humans. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company.

In some embodiments, the pharmaceutical composition of the invention comprises a standard pharmaceutical carrier, such as a sterile phosphate buffered saline solution, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary concentrations of the antigen binding proteins in the formulation may range from about 0.1 mg/ml to about 180 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the antigen binding protein may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the antigen binding protein, may be included in the formulation. Exemplary tonicity agents include polyols, such as mannitol, sucrose or trehalose. In one embodiment the aqueous formulation is isotonic, although hypertonic or hypotonic solutions may be suitable. Exemplary concentrations of the polyol in the formulation may range from about 1% to about 15% w/v.

A surfactant may also be added to the antigen binding protein formulation to reduce aggregation of the formulated antigen binding protein and/or minimize the formation of particulates in the formulation and/or reduce adsorption. Exemplary surfactants include nonionic surfactants such as polysorbates (e.g. polysorbate 20 or polysorbate 80) or poloxamers (e.g. poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. antigen binding protein, buffer, polyol and surfactant) and is essentially free of one or more preservatives, such as benzyl alcohol, phenol, m-cresol, chlorobutanol and benzethonium chloride. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation.

Therapeutic formulations of the bispecific antigen binding protein are prepared for storage by mixing the bispecific antigen binding protein having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In one embodiment, a suitable formulation of the claimed invention contains an isotonic buffer such as a phosphate, acetate, or TRIS buffer in combination with a tonicity agent, such as a polyol, sorbitol, sucrose or sodium chloride, which tonicifies and stabilizes. One example of such a tonicity agent is 5% sorbitol or sucrose. In addition, the formulation could optionally include a surfactant at 0.01% to 0.02% wt/vol, for example, to prevent aggregation or improve stability. The pH of the formulation may range from 4.5-6.5 or 4.5 to 5.5. Other exemplary descriptions of pharmaceutical formulations for antigen binding proteins may be found in US 2003/0113316 and U.S. Pat. No. 6,171,586, each incorporated herein by reference in its entirety.

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Suspensions and crystal forms of antigen binding proteins are also contemplated. Methods to make suspensions and crystal forms are known to one of skill in the art.

The formulations to be used for in vivo administration must be sterile. The compositions of the invention may be sterilized by conventional, well known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying (see Williams and Polli, Journal of Parenteral Science and Technology, Volume 38, Number 2, pages 48-59, 1984). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration (see Chen, Drug Development and Industrial Pharmacy, Volume 18: 1311-1354, 1992).

Excipients have been noted in some cases to act as stabilizers for freeze-dried products (see Carpenter et al., Volume 74: 225-239, 1991). For example, known excipients include polyols (including mannitol, sorbitol and glycerol); sugars (including glucose and sucrose); and amino acids (including alanine, glycine and glutamic acid).

In addition, polyols and sugars are also often used to protect polypeptides from freezing and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the bispecific antigen binding protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing as described herein. Thus, the pharmaceutical formulations may also be formulated for controlled release or for slow release.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

The bispecific antigen binding protein is administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the bispecific antigen binding protein is suitably administered by pulse infusion, particularly with declining doses of the antigen binding protein. In one embodiment the dosing is given by injections, intravenous or subcutaneous injections, depending in part on whether the administration is brief or chronic. Other administration methods are contemplated, including topical, particularly transdermal, transmucosal, rectal, oral or local administration e.g. through a catheter placed close to the desired site. In one embodiment, the antigen binding protein of the invention is administered intravenously in a physiological solution at a dose ranging between 0.01 mg/kg to 100 mg/kg at a frequency ranging from daily to weekly to monthly (e.g. every day, every other day, every third day, or 2, 3, 4, 5, or 6 times per week), a dose ranging from 0.1 to 45 mg/kg, 0.1 to 15 mg/kg or 0.1 to 10 mg/kg at a frequency of once per week, once every two weeks, or once a month.

As used herein, the term “treating” or “treatment” is an intervention performed with the intention of preventing the development or altering the pathology of a disorder. Accordingly, “treatment” refers to both therapeutic treatment and prophylactic or preventative measures. Those in need of treatment include those already diagnosed with or suffering from the disorder or condition as well as those in which the disorder or condition is to be prevented. “Treatment” includes any indicia of success in the amelioration of an injury, pathology or condition, including any objective or subjective parameter such as abatement, remission, diminishing of symptoms, or making the injury, pathology or condition more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, self-reporting by a patient, neuropsychiatric exams, and/or a psychiatric evaluation.

The antigen binding proteins of the invention are useful for detecting target antigen(s) in biological samples and identification of cells or tissues that express the target antigen(s).

The antigen binding proteins described herein can be used for diagnostic purposes to detect, diagnose, or monitor diseases and/or conditions associated with the target antigen(s). Also provided are methods for the detection of the presence of the target antigen(s) in a sample using classical immunohistological methods known to those of skill in the art (e.g., Tijssen, 1993, Practice and Theory of Enzyme Immunoassays, Vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096). The detection of either target can be performed in vivo or in vitro.

Diagnostic applications provided herein include use of the antigen binding proteins to detect expression of target antigen(s). Examples of methods useful in the detection of the presence of the receptor include immunoassays, such as the enzyme linked immunosorbent assay (ELISA) and the radioimmunoassay (RIA).

For diagnostic applications, the antigen binding protein typically will be labeled with a detectable labeling group. Suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, p-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen binding protein via spacer arms of various lengths to reduce potential steric hindrance. Various methods for labeling proteins are known in the art and may be used.

In another embodiment, the bispecific antigen binding protein described herein can be used to identify a cell or cells that express target antigen(s). In a specific embodiment, the antigen binding protein is labeled with a labeling group and the binding of the labeled antigen binding protein to target antigen(s) is detected. In a further specific embodiment, the binding of the antigen binding protein to target antigen(s) is detected in vivo. In a further specific embodiment, the bispecific antigen binding protein is isolated and measured using techniques known in the art. See, for example, Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons.

### EXAMPLES

### Example 1

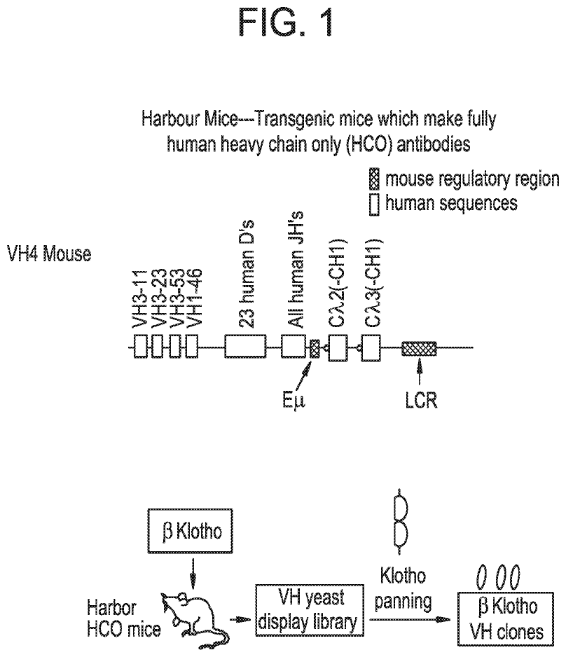

Harbour mice are transgenic mice in which a piece of DNA is integrated in its chromosome (top of FIG. 1). The DNA piece contains 4 different human VH germlines (VH3-11, VH3-23, VH3-53 and VH1-46), 23 D regions, all human JH regions followed with CH2 and CH3 domains of IgG1 antibody. The regulatory element Eμ is inserted between JH and CH2, and LCR (Locus Control Region) is placed downstream of CH3 domain. When Harbour mice are immunized with antigen(s), the immune response will elicit the recombination of V-D-J to form the VH, and produce VH-Fc homodimers. Fc consists of CH2 (Cγ2) -CH3 (Cγ3) domain of human IgG1. During antibody secretion the chaperone BiP binds to CH1 domain until the Light Chain (LC) is correctly folded. LC replaces BiP to bind with CH1, so the CH1 domain is purposely eliminated to allow the secretion of VH-Fc homodimer. The avidity of homodimer and high expression level of Fc-fused molecules confer easy characterization of VH Only (VHO) binders.

The Harbour mice are immunized with the soluble extracellular domain of human beta-Klotho (See bottom portion of FIG. 1). The total RNA is extracted from immune organs (spleen, lymph nodes, bone marrow), mRNAs are amplified by RT-PCR to pull out all VHs. Second round of PCR reaction is carried out to add identical 5′ and 3′ sequences (usually 30-50 bp in length) for homologous recombination inside yeast when co-transformed with yeast display vector which has identical 5′ and 3′ sequences and downstream agglutinin. Agglutinin is a membrane protein for display purpose. The VH fragments are displayed on the yeast surface, and positive VH binders can be fished out by FACS sorting with fluorescent antigen (generally biotin-antigen plus streptavidin-APC). 2-3 rounds of FACS sorting are usually required to enrich the binders before individual yeast colonies can be plated out on agar plates and picked up for further identification.

### Example 2

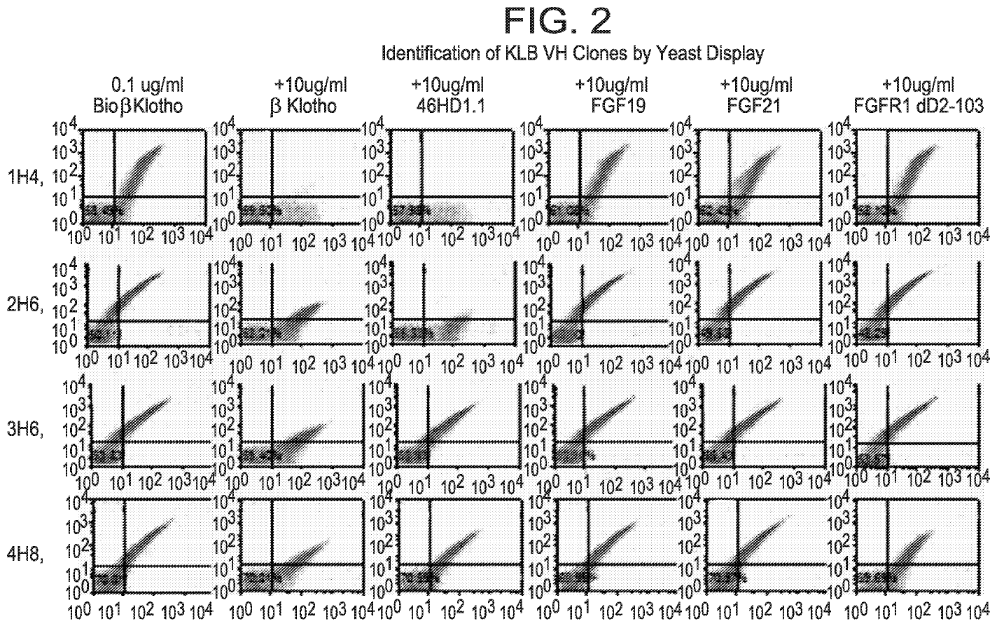

Four types of binders were identified (clones 1H4, 2D5, 3E5, 4H6 in row). After 0.1 μg/mL biotin-beta-Klotho and Streptavidin-APC were added, all VHO clones showed good binding as the majority dots shifted to the up-right quadrum (column 1 of FIG. 2). When 100-fold cold (unlabeled) beta-Klotho was added, the binding of clone 1H4 was completely inhibited whereas binding of clones 2D5 and 3E5 and 4H6 were partially inhibited (column 2 of FIG. 2). When 10 μg/mL of antibody 46D11 (anti-beta Klotho mAb made from Xenomouse) was added, the binding of clone 1H4 was completely inhibited and majority binding of clone 2D5 was inhibited whereas binding of clones 3E5 and 4H5 were not impacted (column 3 of FIG. 2). 10 μg/mL of ligands FGF19 and FGF21 did not compete with the binding of all clones (columns 4 and 5 of FIG. 2). 10 μg/mL of FGFR1c D2-D3 protein did not compete with their bindings either (column 6 of FIG. 2). Accordingly, all VHO clones sorted out by FACS do bind to antigen beta-Klotho and with different affinity as the 100-fold more unlabeled (cold) beta-Klotho can knock down the binding signal at different level. Clone 1H4 shares the same epitope as antibody 46D11, clone 2D5 has a heavily overlapped epitope as that of antibody 46D11, whereas clones 3E5 and 4H6 bind to different epitope comparing with antibody 46D11. All VHO clones (as monomer on yeast) do not compete the binding of ligands FGF19 and FGF21 with beta-Klotho and all VHO clones (as monomer on yeast) do not impact the binding of FGFR1c with beta-Klotho.

### Example 3

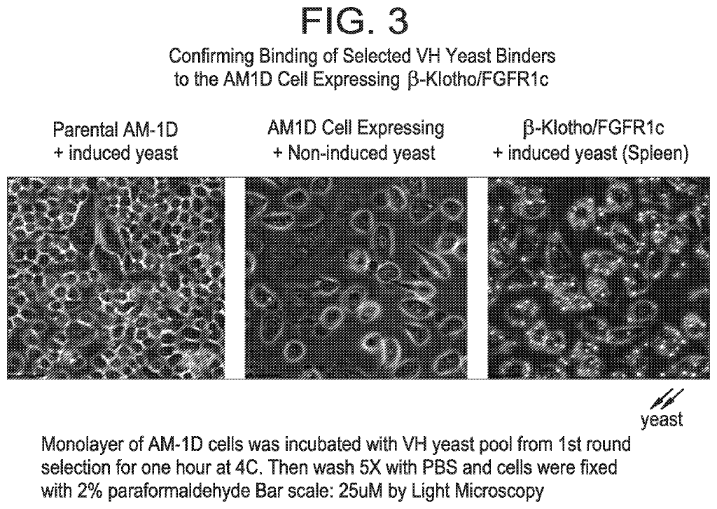

Binding of yeast to beta-Klotho expressed on cell surface was directly observed under microscopy (FIG. 3). Mammalian AM-1D cells stably transfected with human beta-Klotho and FGFR1c or parental AM-1D cells were cultured in 24-well plate and washed with PBS. Non-induced yeast (with no VHO expression) or induced yeast (with VHO expression) in PBS was added for incubation at 4C for 1 hr. Cells were washed 5 times with PBS then fixed with 2% paraformaldehyde. No yeast (small white dots under microscopy) binding to parental AM-1D cells was observed with the addition of induced yeast (left picture) since no beta-Klotho/FGFR1c were expressed on AM-1D cells. The stable AM-1D cells expressing beta-Klotho/FGFR1c did not show any yeast binding with the addition of non-induced yeast (middle picture) whereas so much induced yeast stick to the stable AM-1D cells (right picture). The results indicated that the VHO fragments displayed on yeast surface can bind to the antigen beta-Klotho/FGFR1c complex on stable AM-1D cell surface.

### Example 4

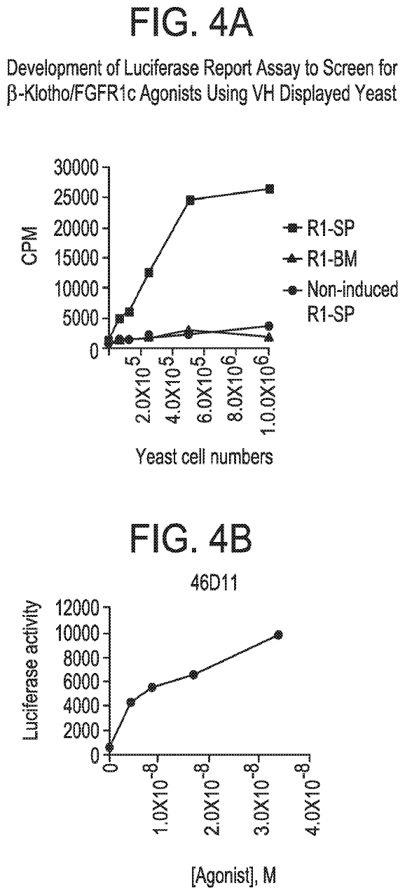

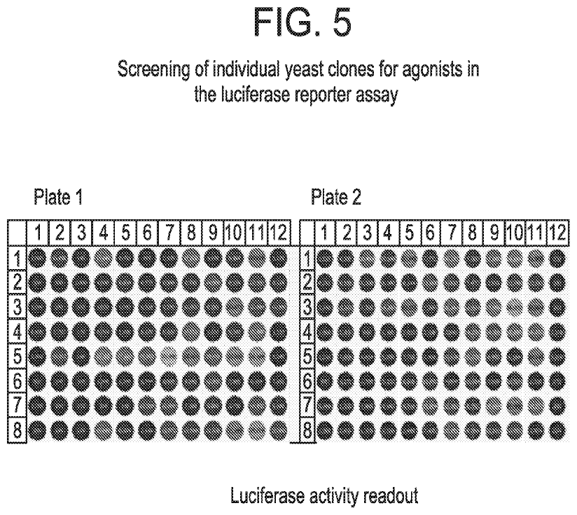

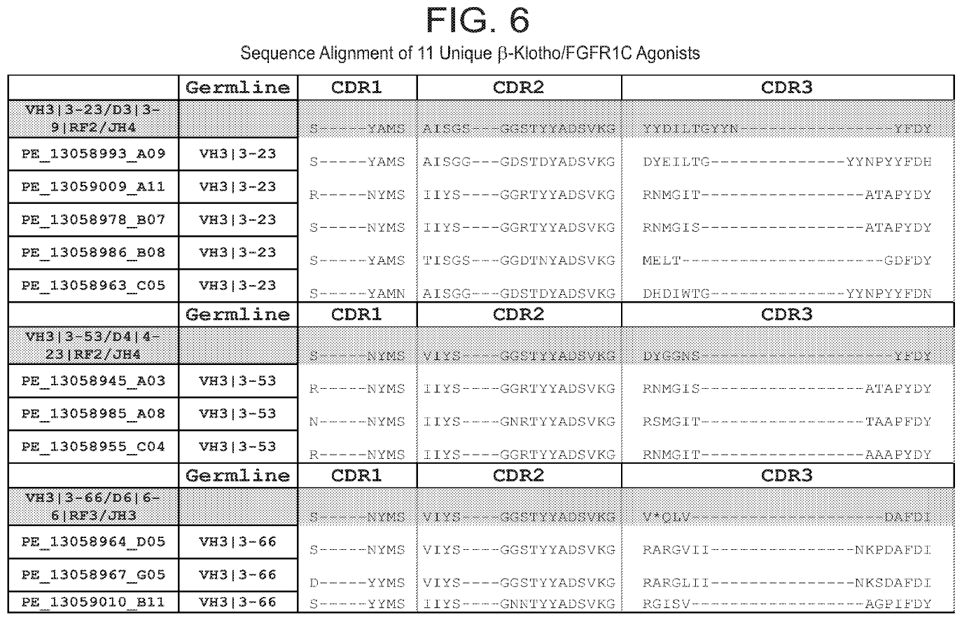

A Steady Glo Luciferase assay was used to screen the VHO pools and binders (left side of FIG. 4). Stable AM-1D cells expressing human beta-Klotho/FGFR1c complex were cultured in 96-well plate in assay media for overnight. On the next day cells were washed with PBS and incubated for 6 hrs with various amount FACS-sorted and induced Round 1 (R1) VHO yeast pools (either from spleen or bone marrow). Non-induced Round 1 (R1) yeast pool from spleen was added as negative control. Cells were lysed then substrates of Steady Glo Luciferase were added to develop blue color. The plate was read and results were recorded in Envision machine. The induced yeast pool from spleen of beta-Klotho immunized Harbor mice caused proliferation of stable AM-1D cells in a dose-dependent manner whereas induced yeast pool from bone marrow or non-induced yeast from spleen did not cause significant proliferation, suggesting that the R1 yeast pool from spleen have abundant VHO binders which can activate βKlotho/FGFR1c complex to proliferate AM-1D cells. The anti-βKlotho antibody 46D11 served as a positive control since it showed dose-dependent proliferation to stable AM-1D cells (right side of FIG. 4).

### Example 5

Two 96-w plates of individual yeast colonies (192) were grown in yeast culture medium and induced at 30° C. for 3 days. Steady Glo Luciferase assay was used to screen βKlotho agonists. Around 50% of colonies can agonize the βKlotho/FGFR1c complex and proliferate stable AM-1D cells. The purple color indicates positive proliferation signal while blue color (baseline) indicates no proliferation.

### Example 6

Alignment of amino acids in CDR loops of 11 unique beta-Klotho VHO binders. Five unique VHO binders are classified in VH3-23 germline, 3 unique VHO binders in VH3-53 germline, and 3 unique VHO binders in VH3-66 germline (sequencewise close to VH3-11). Each unique binder has different amino acid sequence in CDR loops, especially in CDR3.

### Example 7

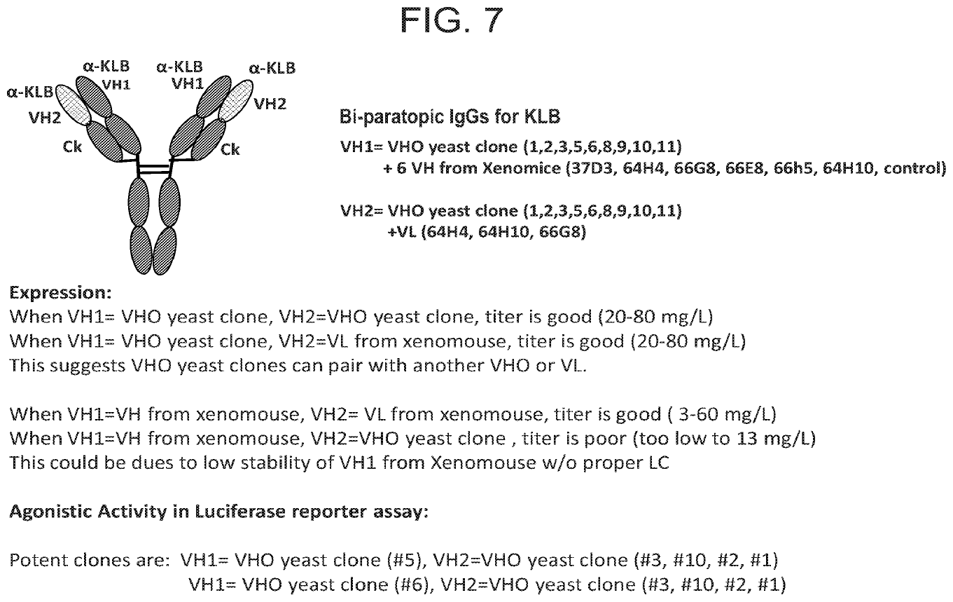

This example describes a biparatopic IgG antibody in which 2 different VHs are linked to CH1 and CL respectively was explored to assess the activation of βKlotho/FGFR1c complex. The DNA for the first VH (VHO clones 1, 2, 3, 5, 6, 8, 9, 10 from Harbour mice (amino acid SEQ ID NOs: 1-8; DNA SEQ ID NOs: 15-22) and VH from βKlotho immunized Xenomouse antibody clones 37D3, 64H4, 66G8, 66E8, 66H5, 64H10 (amino acid SEQ ID NOs: 9-14; DNA SEQ ID NOs: 23-28) and an control antibody were fused at N-terminus with DNA encoding CH1-hinge-CH2-CH3 of human IgG1, the DNA for the second VH (VHO clones 1, 2, 3, 5, 6, 8, 9, 10 from Harbour mice or standard VL from βKlotho immunized Xenomouse antibody (clones 64H4, 64H10 and 66G8) was linked at N-terminus of Cu. The plasmids were co-transfected by matrix combinations (15*12=180) into mammalian 2936E cells in 96 deep well plates. The supernant was harvested and Fc titers were measured by ForteBio Octet Red 96. The supernant was then assessed/screened for the activation of βKlotho/FGFR1c complex on stable AM-1D cells. Expression and activity results are shown in below FIG. 7.

### Example 8

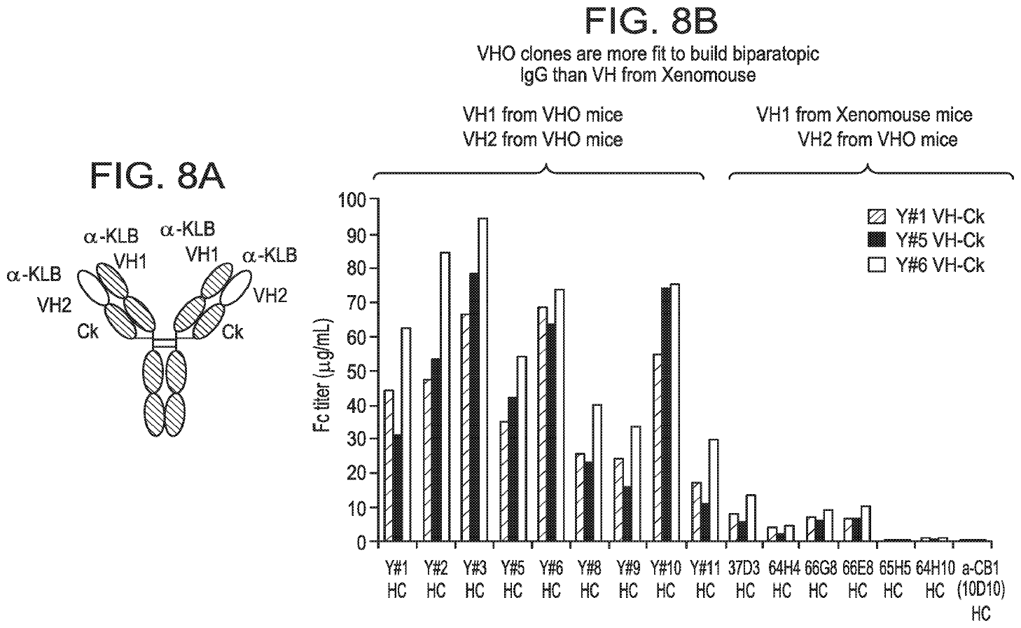

FIG. 8 shows the Fc titer of anti-βKlotho biparatopic antibodies. The left figure is the depiction of biparatopic configuration and the right figure is the Fc titer for different combinations. When the VH1 in HC is coming from VHO of Harbour mice and VH2 in LC is also coming from VHO Harbour mice, the biparatopic antibodies are generally expressing very well. However, when the VH1 in HC is coming from Xenomouse mice and VH2 in LC is coming from VHO Harbour mice, the biparatopic antibodies are expressing poorly. The results indicated that VHOs from Harbour mice are stable for expression since all VHOs are pre-selected in vivo in Harbour mice, only the VHOs with good solubility and stability can be matured and secreted.

### Example 9

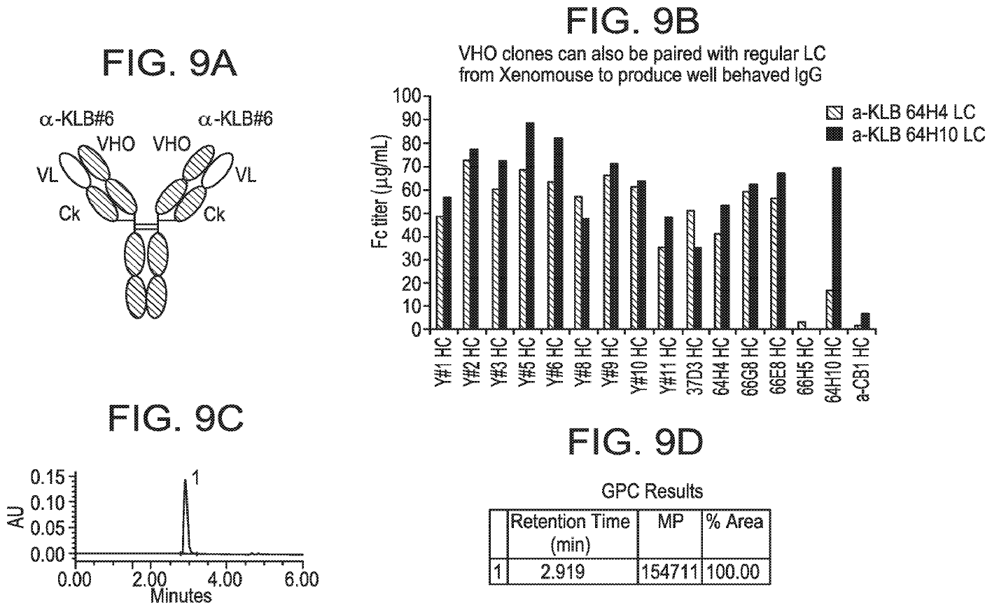

FIG. 9 shows the Fc titer of anti-βKlotho VHOs when expressed as standard antibodies. The left figure is the depiction of biparatopic configuration and the right figure is the Fc titers of VHOs when expressed as standard IgG. When the 9 different VHOs (#1, 2, 3, 5, 6, 8, 9, 10, 11) from Harbour mice were cloned into the HC, then co-transfected with a standard Kappa LC (from anti-βKlotho xenomouse clone 64H4), all antibodies were expressed very well. Similarly, When the 9 different VHOs (#1, 2, 3, 5, 6, 8, 9, 10, 11) from Harbour mice were cloned into the HC, then co-transfected with a standard Lambda LC (from anti-βKlotho xenomouse clone 64H10), all antibodies were also expressed very well, when comparing with the standard HCs from anti-βKlotho Xenomouse clones 37D3, 64H4, 66G8, 66E8, 66H5, 64H10. Notably, HC of clone 64H10 prefers its own LC, whereas HC of clone 64H4 can tolerate other LC. The internal control anti-CB1 HC did not express well when co-transfected with other LCs. The results indicated that VHOs from Harbour mice are versatile to be expressed as standard IgG antibodies and biparatopic antibodies (see above) since VHOs are pre-selected in vivo in Harbour mice, only the VHOs with good solubility and stability can be matured and secreted. The bottom portion of FIG. 9 shows the analytical SEC profile of one standard antibody configuration in which VHO was subcloned in HC, 100% sharp main peak, indicating the good purification profile.

### Example 10

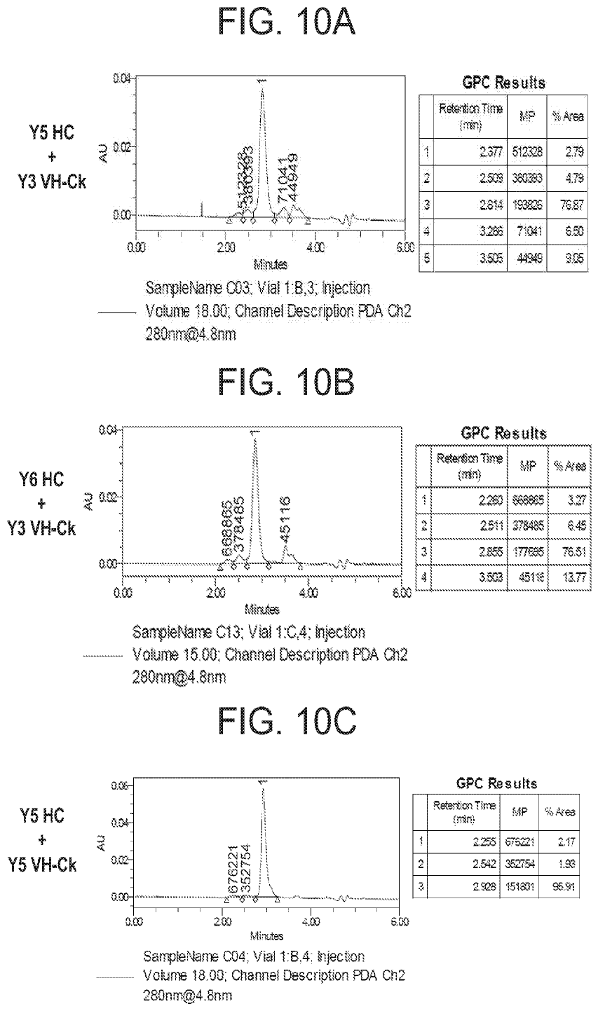

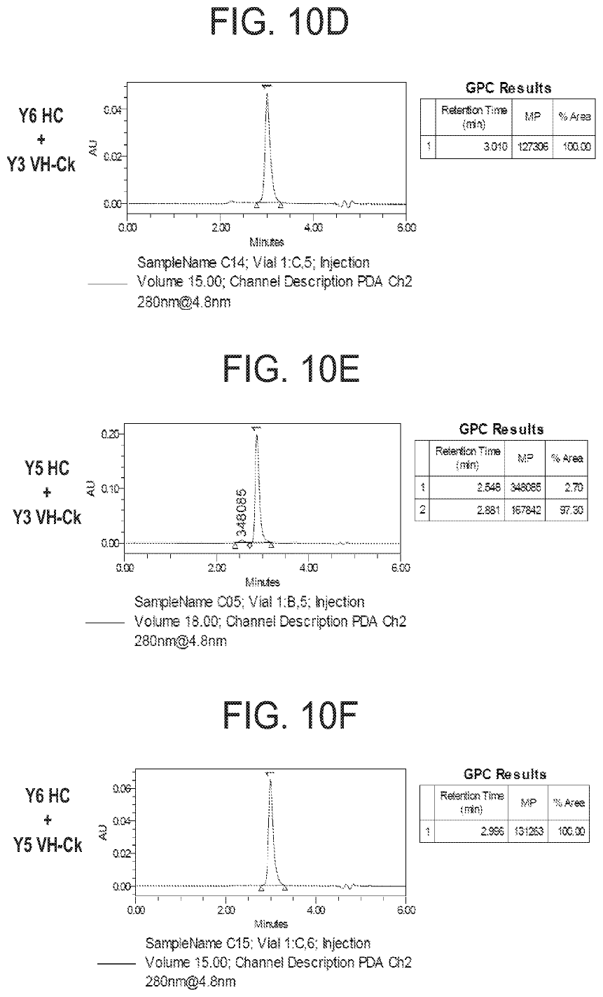

FIG. 10 show the purification profiles of expressed bi-paratopic antibodies: Left panel, top: the biparatopic Ab of VHO #5 in HC pairing with VHO #3 in LC.

Left panel, middle: the biparatopic Ab of VHO #5 in HC pairing with VHO #5 itself in LC.

Left panel, bottom: the biparatopic Ab of VHO #5 in HC pairing with VHO #6 in LC.

Right panel, top: the biparatopic Ab of VHO #6 in HC pairing with VHO #3 in LC.

Right panel, middle: the biparatopic Ab of VHO #6 in HC pairing with VHO #5 in LC.

Right panel, bottom: the biparatopic Ab of VHO #6 in HC pairing with VHO #6 itself in LC.

In summary, the results showed that VHO clones #5 and #6 are good modules for biparatopic expression whether or not they are paired with its own VHO in LC.

### Example 11

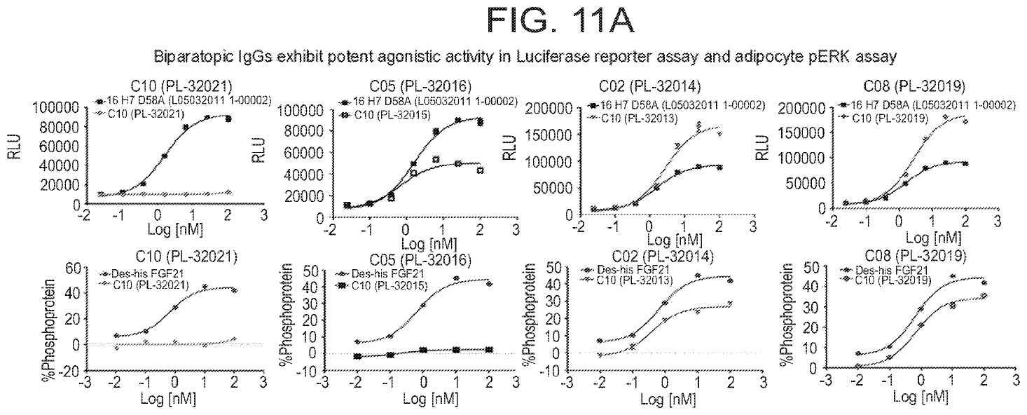

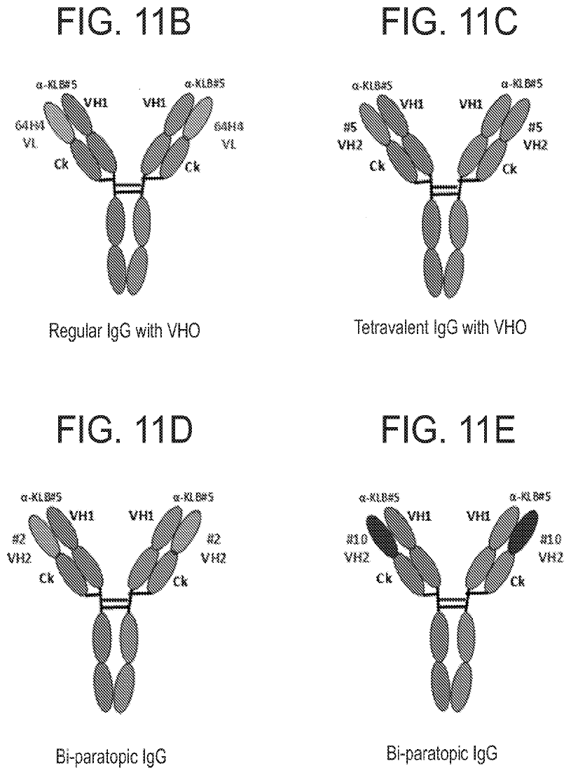

FIG. 11 shows the function screen of top biparatopic antibodies. The Luciferase report assay (top row) and adipocyte pERK assay (middle row) were used to screen biparatopic antibodies. Luciferase reporter assay is a primary cell-based assay for screen since it is faster and cheaper. The adipocyte pERK is a physiological cell-based function assay, good for activity confirmation of top antibody clones.

The bottom row are configuration (and components) of different antibodies.

The 1^{st} column: sample C10 (protein lot no. PL-32021) is a standard antibody configuration. The VHO #5 from Harbour mice in HC was co-transfected with a standard LC which has VL from anti-βKlotho clone 64H4 and C-kappa constant domain. This protein (gray curve) did not show any activity in both assays whereas the positive control FGF21 was active in both assays. The 1^{st} column serves as internal and negative control.

The 2^{nd} column: sample C05 (protein lot no. PL-32016) is a biparatopic antibody configuration. The VHO #5 from Harbour mice in HC was co-transfected with a LC which has VHO #5 itself from Harbour mice and downstream C-kappa constant domain. This protein (blue curve) showed some activity in Luciferase reporter assay whereas the positive control FGF21 was very active. However, in adipocyte pERK assay, this protein C05 did not show any activity.

The 3^{rd} column: sample C02 (protein lot no. PL-32014) is a biparatopic antibody configuration. The VHO #5 from Harbour mice in HC was co-transfected with a LC which has a different VHO #2 from Harbour mice and downstream C-kappa constant domain. This protein (green curve) showed much higher activity in Luciferase reporter assay than the positive control FGF21, in adipocyte pERK assay this protein C02 showed decent activity.

The 4^{th} column: sample C08 (protein lot no. PL-32019) is a biparatopic antibody configuration. The VHO #5 from Harbour mice in HC was co-transfected with a LC which has a different VHO #10 from Harbour mice and C-kappa constant domain. This protein (green curve) showed much higher activity in Luciferase reporter assay than the positive control FGF21, and in adipocyte pERK assay this protein C02 showed very good activity (very close to positive control FGF21).

VHOs #5, #2 and #10 bind to different epitope on p-Klotho by competition ELISA. These results suggested that 2 different VHOs could simultaneously bind to 2 different epitopes, stabilize certain active conformations. The bi-valency of biparatopic Ab may cross-link and activate the β-Klotho/FGFR1c complex.

### Example 12

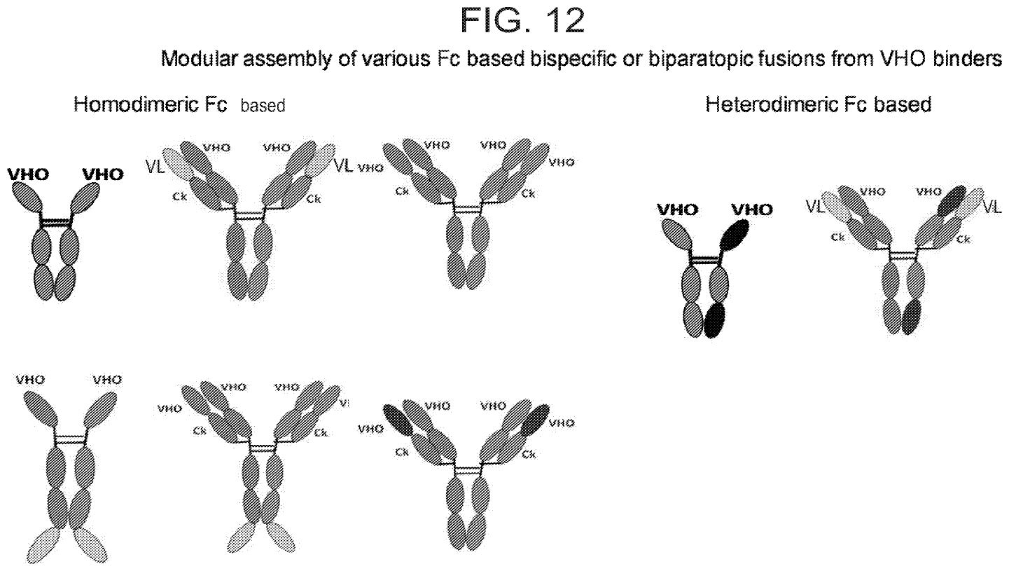

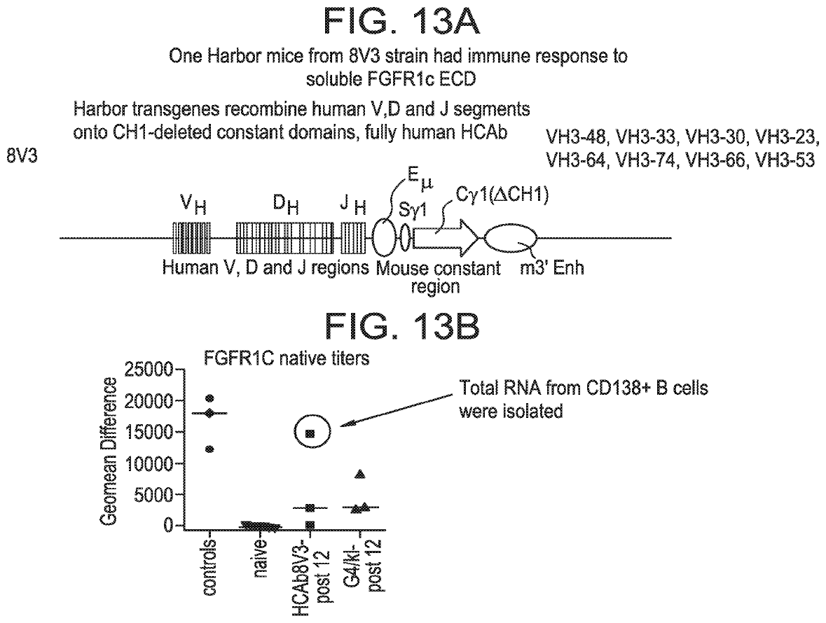

The VHo modules can be explored as different formats. FIG. 12 (top row) shows mono-specific Fc fusion (homodimer), standard IgG antibody, biparatopic antibody, bi-specific Fc fusion (heterodimer), and a bi-specific heterodimeric antibody. FIG. 12 (bottom row) shows bispecific homodimeric VHO-Fc-VHO, VHO-tailed bi-paratopic antibody, and bi-specific bi-paratopic antibody. Different colors mean different VHO modules (not itself) or charge engineered CH3 domain.

### Example 13

Another Harbour mice strain 8V3 was also utilized and evaluated. Eight different VH germlines (H3-48, VH3-33, VH3-30, VH3-23, VH3-64, VH3-74, VH3-66 and VH3-53) and all D region and J regions followed by regulatory element Ep, mouse Fc (without CH1 domain as described in FIG. 1 at the beginning) and 3′ enhancer are integrated in mouse genome. This strain was immunized with human FGFR1c, one mouse showed good antibody titer, and the total RNA from this mouse was isolated from plasma cells (CD138 positive), then VHOs were amplified by RT-PCR.

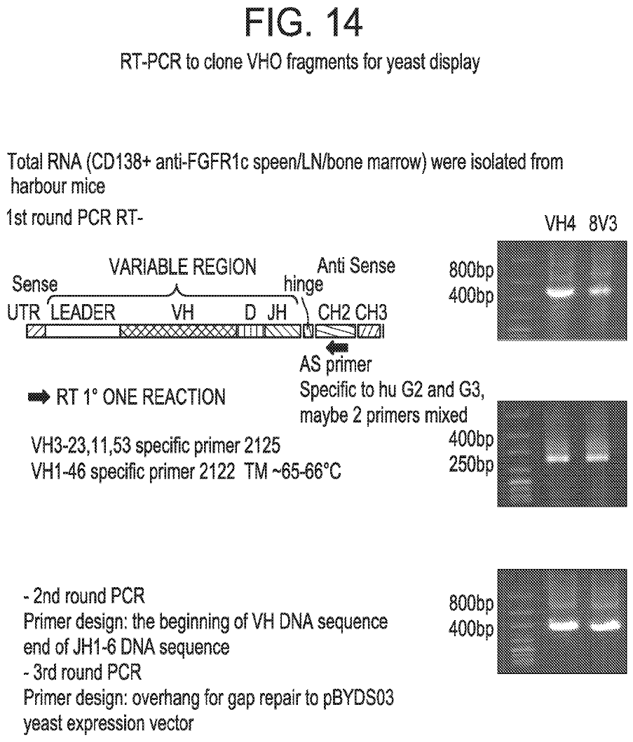

FIG. 14 shows how RT-PCR was used to clone VHO fragments for yeast display. Three rounds of PCR reactions were carried out to pull out and amplify the FGFR1c VHO fragments. After Reverse Transcription (RT) reaction, VH-specific forward (or sense) primers (oligo number 2125 (SEQ ID NO: 29) and 2122 (SEQ ID NO: 30)) and reverse primer (or anti-sense primer, AS (SEQ ID NO: 31)) located in mouse CH2 domain were used for the 1^{st} round PCR, the products are around 600 bp. VH-specific forward (or sense) primers (oligo number 2125 and 2122) and reverse primer (or anti-sense primer, AS) located in mouse JH were used for the 2^{nd} round PCR. In this way, we got more specific VHO DNA products which are around 350 bp (shorter than those of 1^{st} PCR product because of internal PCR strategy). For the 3^{rd} round of PCR reactions, primers with identical DNA sequence in yeast display vector pBYDS03 were used to get final PCR products which have the same and short (˜30 bp) for homologous recombination to construct yeast display libraries.

### Example 14

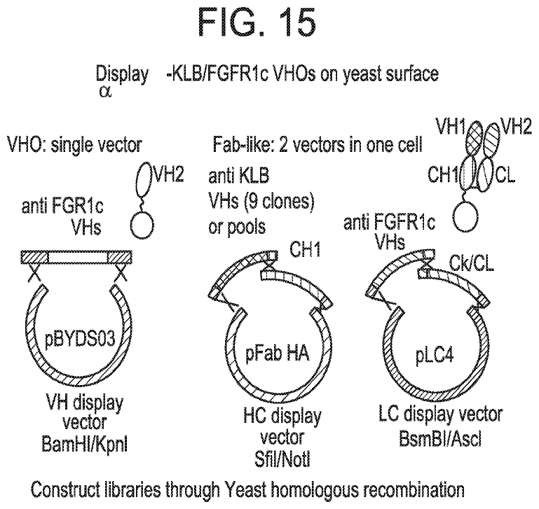

FIG. 15 (left) shows the anti-FGFR1c VHO modules can be displayed as single domain on yeast surface when linked with display protein agglutinin. FIG. 15 (right) shows the anti-βKlotho VHO modules are fused with CH1 domain of antibody and linked with agglutinin for display. When coupled to anti-FGFR1c VHO and C-kappa domain in a separate vector, two types of VHOs targeting βKlotho and FGFR1c separately can be displayed on yeast surface as Fab-like format for the identification of bi-specific antibodies.

### Example 15

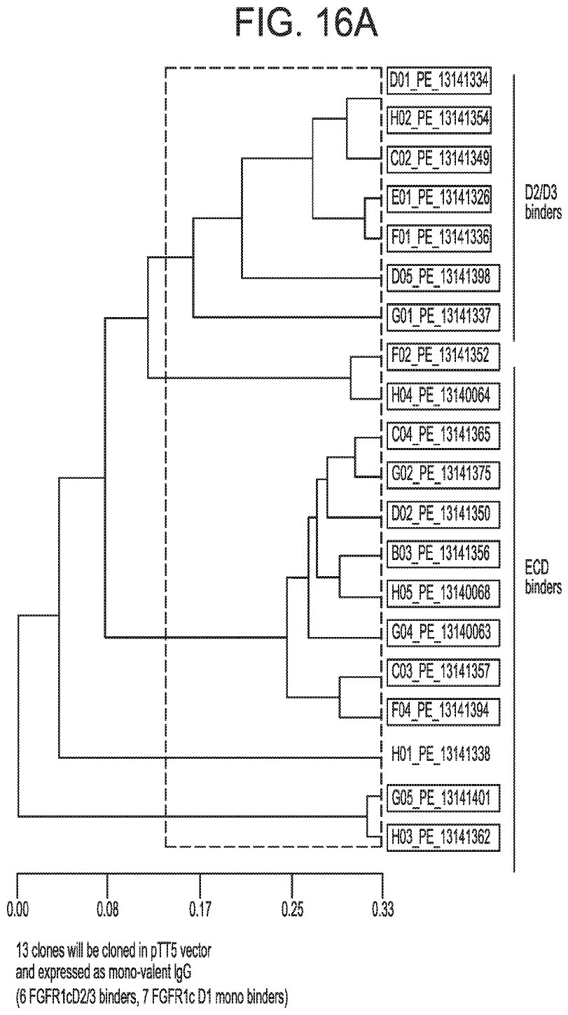

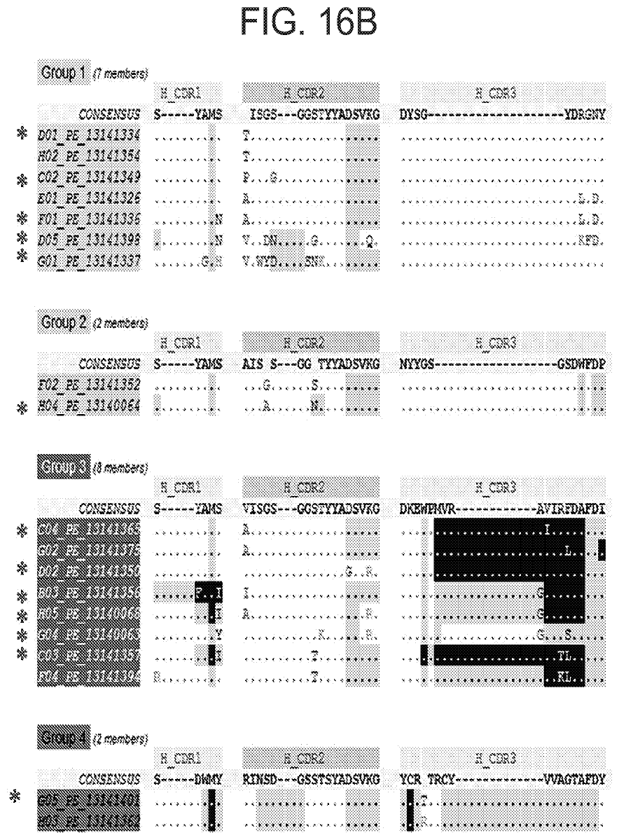

FIG. 16 shows 20 unique anti-FGFR1c VHOs identified by yeast display. The VHOs are clustered in 4 different groups, mainly based on their CDR3 loop length and residue sequences. 7 out of 20 unique VHOs bind to D2-D3 of FGFR1c by plate ELISA while other 13 unique VHOs only bind to full length ECD of FGFR1c. 13 interesting VHOs (marked with a star symbol) were chosen for convention to human IgG and expression in mammalian cells.

### Example 16

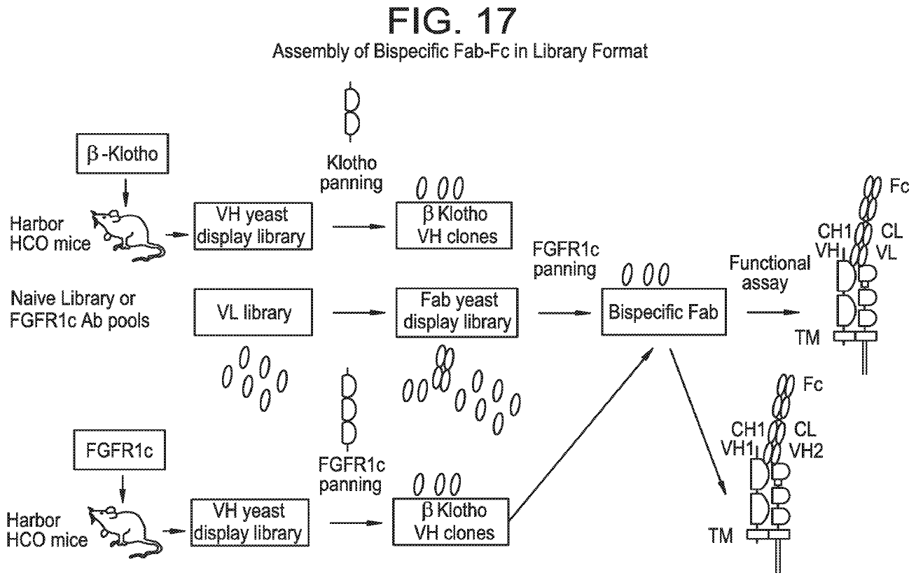

FIG. 17 shows an alternative way to make anti-βKlotho/FGFR1c bispecific antibody libraries on yeast surface. The anti-βKlotho VHO modules are fused with CH1 domain of antibody and linked with display protein agglutinin. When co-transfected with anti-FGFR1c VLs either from naïve LC library or from FGFR1c immunized Xenomouse and downstream C-kappa domain in a separate vector, the VHO targeting βKlotho and VL targeting FGFR1c can be displayed on yeast surface as Fab-like format for screening. The bi-specific antibodies as Fc fusion format can be generated later on (top of figure). The anti-βKlotho VHO modules are fused with CH1 domain of antibody and linked with display protein agglutinin. When co-transfected with anti-FGFR1c VHOs from FGFR1c immunized Harbour mice and downstream C-kappa domain in a separate vector, the VHOs targeting βKlotho and FGFR1c separately can be displayed on yeast surface as Fab-like format for screening. The bi-specific antibodies as Fc fusion format can be generated later on. (bottom of figure).

Sequence Listing
Sequence Listing
VHO1
SEQ ID NO: 1
EVQLLETGGGLIQPGGSLRLSCAASGFNVSRNYMSWVRQAPGKGLEWVSI
IYSGGRTYYADSVKGRFTISRDNSKNMLYLQMNSLSAEDTAVYYCAKRNM
GISATAPYDYWGQGTLVTVSRTVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGEC
SEQ ID NO: 15
GAGGTGCAGCTGTTGGAGACTGGAGGAGGCCTGA
TCCAGCCTGGGGGGTCCCTGAGACTCTCCTGTGCAGCCTCTGGGTTCAAC
GTCAGTCGCAACTATATGAGTTGGGTCCGCCAGGCTCCAGGGAAGGGGCT
GGAGTGGGTCTCAATTATTTATAGCGGTGGTAGAACATACTACGCAGACT
CCGTGAAGGGCCGATTCACCATCTCCAGAGACAATTCCAAGAATATGCTG
TATCTTCAAATGAACAGCCTGAGTGCCGAGGACACGGCCGTTTATTACTG
TGCGAAAAGGAATATGGGTATATCAGCAACTGCCCCATATGACTACTGGG
GCCAGGGAACCCTGGTCACCGTCTCCCGTACGGTGGCTGCACCATCTGTC
TTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAG
CAGGACAGCAAGGACAGCACCTACAGCCTCAGCAGCACCCTGACGCTGAG
CAAAGCAGACTAGGAGAAACACAAAGTCTACGCCTGCGAAGTCACCCATC
AGGGCCTGAGCTCGCCCGTCACAAAGAGCTTCAACAGGGGAGAGTGT
VHO2
SEQ ID NO: 2
EVQLVETGGGLIQPGGSLRLSCAASGFNVSRNYMSWVRQAPGKGLEWVSI
IYSGGRTYYADSVKGRFTISRDNSKNMLYLQMNSLRAEDTAVYYCAKRNM
GITAAAPYDYWGQGTLVTVSRTVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGEC
SEQ ID NO: 16
GAGGTGCAGCTGGTGGAGACTGGAGGAGGCCTGA
TCCAGCCTGGGGGGTCCCTGAGACTCTCCTGTGCAGCCTCTGGGTTCAAC
GTCAGTCGCAACTATATGAGCTGGGTCCGCCAGGCTCCAGGGAAGGGGCT
GGAGTGGGTCTCAATTATTTATAGCGGTGGTAGAACATACTACGCAGACT
CCGTGAAGGGCCGATTCACCATCTCCAGAGACAATTCCAAGAATATGCTG
TATCTTCAAATGAACAGCCTGAGAGCCGAGGACACGGCCGTTTATTACTG
TGCGAAAAGGAATATGGGTATAACAGCAGCTGCCCCGTATGACTACTGGG
GCCAGGGAACCCTGGTCACCGTCTCCCGTACGGTGGCTGCACCATCTGTC
TTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAG
CAGGACAGCAAGGACAGCACCTACAGCCTCAGCAGCACCCTGACGCTGAG
CAAAGCAGACTAGGAGAAACACAAAGTCTACGCCTGCGAAGTCACCCATC
AGGGCCTGAGCTCGCCCGTCACAAAGAGCTTCAACAGGGGAGAGTGT
VHO3
SEQ ID NO: 3
EVQLLESGGGLVQPGGSLRLSCAASGFNVSRNYMSWVRQAPGKGLEWVSI
IYSGGRTYYADSVKGRFTISRDNSKNTLYLQMNSLRAEDTAVYYCAKRNM
GITATAPYDYWGQGTLVTVSRTVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGEC
SEQ ID NO: 17
GAGGTGCAGCTGTTGGAGTCTGGGGGAGGCTTGG
TACAGCCTGGGGGGTCCCTGAGACTCTCCTGTGCAGCCTCTGGGTTCAAC
GTCAGTCGCAACTATATGAGCTGGGTCCGCCAGGCTCCAGGGAAGGGGCT
GGAGTGGGTTTCAATTATTTATAGCGGTGGTAGAACATACTACGCAGACT
CCGTGAAGGGCCGGTTCACCATCTCCAGAGACAATTCCAAGAACACGCTG
TATCTTCAAATGAACAGCCTGAGAGCCGAGGACACGGCCGTTTATTACTG
TGCGAAAAGGAATATGGGTATAACAGCAACTGCCCCGTATGACTACTGGG
GCCAGGGAACCCTGGTCACCGTCTCCCGTACGGTGGCTGCACCATCTGTC
TTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAG
CAGGACAGCAAGGACAGCACCTACAGCCTCAGCAGCACCCTGACGCTGAG
CAAAGCAGACTAGGAGAAACACAAAGTCTACGCCTGCGAAGTCACCCATC
AGGGCCTGAGCTCGCCCGTCACAAAGAGCTTCAACAGGGGAGAGTGT
VHO5
SEQ ID NO: 4
QVQLVESGGGLVKPGGSLRLSCAASGFTFSSYAMSWVRQAPGKGLEWVSA
ISGGGDS TDYADSVKGRFTISRDNSKNTLYLQMNSLRAEDTAVYYCAKDY
EILTGYYNPYYFDHWGQGTLVTVSRTVAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGEC
SEQ ID NO: 18
CAGGTGCAGCTGGTGGAGTCTGGGGGAGGCTTGG
TCAAGCCTGGAGGGTCCCTGAGACTCTCCTGTGCAGCCTCTGGATTCACC
TTTAGCAGCTATGCCATGAGCTGGGTCCGCCAGGCTCCAGGGAAGGGGCT
GGAGTGGGTCTCAGCTATTAGTGGTGGTGGTGATAGCACAGACTACGCAG
ACTCCGTGAAGGGCCGGTTCACCATCTCCAGAGACAATTCCAAGAACACG
CTGTATCTGCAAATGAACAGCCTGAGAGCCGAGGACACGGCCGTATATTA
CTGTGCGAAAGATTAGGAGATTTTGACTGGTTATTATAACCCGTACTACT
TTGACCACTGGGGCCAGGGAACCCTGGTCACCGTCTCCCGTACGGTGGCT
GCACCATCTGTCTTCATCTTCCCGCCATCTGATGAGCAGTTGAAATCTGG
AACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAG
AGTGTCACAGAGCAGGACAGCAAGGACAGCACCTACAGCCTCAGCAGCAC
CCTGACGCTGAGCAAAGCAGACTACGAGAAACACAAAGTCTACGCCTGCG
AAGTCACCCATCAGGGCCTGAGCTCGCCCGTCACAAAGAGCTTCAACAGG
GGAGAGTGT
VHO6
SEQ ID NO: 5
EVQLVESGGGLVQPGGSLRLSCAASGFTESSYAMNWVRQAPGKGLEWVSA
ISGGGDSTDYADSVKGRFTISRDNSKNTLYLQMNSLRAEDTAVYYCAKDH
DIWTGYYNPYYFDNWGQGTLVTVSRTVAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGEC
SEQ ID NO: 19
GAGGTGCAGCTGGTGGAGTCTGGGGGAGGCTTGG
TACAGCCTGGGGGGTCCCTGAGACTCTCCTGTGCAGCCTCTGGATTCACC
TTTAGCAGCTATGCCATGAACTGGGTCCGCCAGGCTCCAGGGAAGGGGCT
GGAGTGGGTCTCAGCTATTAGTGGTGGTGGTGATAGCACAGACTACGCAG
ACTCCGTGAAGGGCCGGTTCACCATCTCCAGAGACAATTCCAAGAACACG
CTGTATCTGCAAATGAACAGCCTGAGAGCCGAGGACACGGCCGTATATTA
CTGTGCGAAAGATCACGATATTTGGACTGGTTATTATAACCCGTACTACT
TTGACAACTGGGGCCAGGGAACCCTGGTCACTGTCTCCCGTACGGTGGCT
GCACCATCTGTCTTCATCTTCCCGCCATCTGATGAGCAGTTGAAATCTGG
AACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAG
AGTGTCACAGAGCAGGACAGCAAGGACAGCACCTACAGCCTCAGCAGCAC
CCTGACGCTGAGCAAAGCAGACTACGAGAAACACAAAGTCTACGCCTGCG
AAGTCACCCATCAGGGCCTGAGCTCGCCCGTCACAAAGAGCTTCAACAGG
GGAGAGTGT
VHO8
SEQ ID NO: 6
QVQLVESGGGLVKPGGSLRLSCAASGFTVNSNYMSWVRQAPGKGLEWVSV
IYSGGSTYYADSVKGRFTISRDNSKNTLYLQMNSLRAEDTAVYYCARRAR
GVIINKPDAFDIWGQGTMVTVSRTVAAPSVFIFPPSDEQLKSGTASWCL
LNNFYPREAKVQWKVDNALQSGNSQESVTEQDSKDSTYSLSSTLTLSKAD
YEKHKVYACEVTHQGLSSPVTKSFNRGEC
SEQ ID NO: 20
CAGGTGCAGCTGGTGGAGTCTGGGGGAGGCTTGG
TCAAGCCTGGAGGGTCCCTGAGACTCTCCTGTGCAGCCTCTGGGTTCACC
GTCAATAGCAACTACATGAGCTGGGTCCGCCAGGCTCCAGGGAAGGGGCT
GGAGTGGGTCTCAGTTATTTATAGCGGTGGTAGCACATACTACGCAGACT
CCGTGAAGGGCCGATTCACCATCTCCAGAGACAATTCCAAGAACACGCTG
TATCTTCAAATGAACAGCCTGAGAGCCGAGGACACGGCCGTATATTACTG
TGCGAGAAGGGCTCGGGGAGTTATTATAAACAAACCTGATGCTTTTGATA
TCTGGGGCCAAGGGACAATGGTCACCGTCTCCCGTACGGTGGCTGCACCA
TCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTC
ACAGAGCAGGACAGCAAGGACAGCACCTACAGCCTCAGCAGCACCCTGAC
GCTGAGCAAAGCAGACTACGAGAAACACAAAGTCTACGCCTGCGAAGTCA
CCCATCAGGGCCTGAGCTCGCCCGTCACAAAGAGCTTCAACAGGGGAGAG
TGT
VHO9
SEQ ID NO: 7
QVQLVESGGGLVQPGGSLRLSCAASGFTFSDYYMSWVRQAPGKGLEWVSV
IYSGGSTYYADSVKGRFTISRDNSKNTLYLQMNSLRAEDTAVYYCARRAR
GLIINKSDAFDIWGQGTMVTVSRTVAAPSVFIFPPSDEQLKSGTASVVCL
LNNFYPREAKVQWKVDNALQSGNSQESVTEQDSKDSTYSLSSTLTLSKAD
YEKHKVYACEVTHQGLSSPVTKSFNRGEC
SEQ ID NO: 21
CAGGTGCAGCTGGTGGAGTCTGGGGGAGGCTTGG
TACAGCCTGGGGGGTCCCTGAGACTCTCCTGTGCAGCCTCTGGATTCACC
TTCAGTGACTACTACATGAGCTGGGTCCGCCAGGCTCCAGGGAAGGGGCT
GGAGTGGGTCTCAGTTATTTATAGCGGTGGTAGCACATACTACGCAGACT
CCGTGAAGGGCCGATTCACCATCTCCAGAGACAATTCCAAGAACACGCTG
TATCTGCAAATGAACAGCCTGAGAGCCGAGGACACGGCCGTGTATTACTG
TGCGAGAAGGGCTCGGGGACTTATTATAAACAAATCTGATGCTTTTGATA
TCTGGGGCCAAGGGACAATGGTCACCGTCTCCCGTACGGTGGCTGCACCA
TCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTC
ACAGAGCAGGACAGCAAGGACAGCACCTACAGCCTCAGCAGCACCCTGAC
GCTGAGCAAAGCAGACTACGAGAAACACAAAGTCTACGCCTGCGAAGTCA
CCCATCAGGGCCTGAGCTCGCCCGTCACAAAGAGCTTCAACAGGGGAGAG
TGT
VHO10
SEQ ID NO: 8
EVQLVESGGGLVKPGGSLRLSCAASGFTVSSYYMSWVRQAPGKGLEWVSI
IYSGNNTYYADSVKGRFTISRDNSKNTLYLQMNSLRVEDTAVYYCARRGI
SVAGPIFDYWGQGTLVTVSRTVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGEC
SEQ ID NO: 22
GAGGTGCAGCTGGTGGAGTCTGGGGGAGGCTTGG
TCAAGCCTGGGGGGTCCCTGAGACTCTCCTGTGCAGCCTCTGGGTTCACC
GTCAGTAGCTACTACATGAGCTGGGTCCGCCAGGCTCCAGGGAAGGGGCT
GGAGTGGGTCTCAATTATTTATAGCGGTAATAACACATACTACGCAGACT
CCGTGAAGGGCCGATTCACCATCTCCAGAGACAATTCCAAGAACACGCTG
TATCTTCAAATGAACAGCCTGAGAGTCGAGGACACGGCCGTCTATTACTG
TGCGAGAAGAGGTATATCAGTGGCTGGTCCCATCTTTGACTATTGGGGCC
AGGGAACCCTGGTCACCGTCTCCCGTACGGTGGCTGCACCATCTGTCTTC
ATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAG
GACAGCAAGGACAGCACCTACAGCCTCAGCAGCACCCTGACGCTGAGCAA
AGCAGACTACGAGAAACACAAAGTCTACGCCTGCGAAGTCACCCATCAGG
GCCTGAGCTCGCCCGTCACAAAGAGCTTCAACAGGGGAGAGTGT
37D3 VH
SEQ ID NO: 9
EVQLVESGGGLAKPGGSLRLSCAASGETFRNAWMSWVRQAPGKGLEWVGR
IKSKTDGGTTDYAAPVKGRFTISRDDSKNTLYLQMNSLKTEDTAEYYCIT
DRVLSYYAMAVWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSC
SEQ ID NO: 23
GAGGTCCAGCTGGTGGAGAGCGGAGGTGGACTCG
CCAAGCCGGGTGGTTCTCTGAGGCTGAGCTGTGCCGCCTCCGGCTTCACA
TTCAGGAACGCCTGGATGAGCTGGGTTAGGCAAGCTCCAGGTAAAGGCCT
CGAATGGGTCGGCCGCATCAAAAGCAAGACTGATGGTGGAACCACAGACT
ACGCCGCTCCTGTTAAGGGACGCTTCACAATTAGTCGTGATGATTCCAAG
AATACCCTGTACCTGCAGATGAACTCTCTGAAGACAGAAGACACAGGAGA
GTATTATTGCATTACTGACCGTGTGCTGTCCTACTACGCCATGGCTGTGT
GGGGCCAGGGAACCACTGTTACCGTGAGCTCTGCTAGCACCAAGGGCCCA
TCCGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTC
CAGCAGCTTGGGCACCCAGACCTACATCTGCAACGTGAATCACAAGCCCA
GCAACACCAAGGTGGACAAGAGAGTTGAGCCCAAATCTTGT
64H4
SEQ ID NO: 10
QVQLVQSGAEVKKPGASVKVSCRASGYTFTSFDINWVRQATGQGLEWMGW
MNPNSGNTDYAQKFQGRVTMTRNTSISTAYMELSDLRSEDTAVYFCARGG
SWHYYFYYGLDVWGQGTTVTVSSASTKGPSVFPLAPSSKSTSGGTAALGC
LVKDYFPEPVTVSWNSGALTSGVHTFPAVLQSSGLYSLSSWTVPSSSLG
TQTYICNVNHKPSNTKVDKRVEPKSC
SEQ ID NO: 24
CAGGTCCAACTGGTTCAGTCTGGCGCCGAGGTGA
AGAAGCCCGGAGCCAGCGTGAAAGTTTCCTGCCGGGCCTCCGGGTACACC
TTTACCAGTTTCGATATCAACTGGGTGCGCCAGGCCACAGGACAGGGTTT
GGAATGGATGGGTTGGATGAACCCTAACAGTGGTAACACTGATTATGCTC
AAAAATTCCAAGGCCGCGTTACCATGACCAGAAACACCAGTATTTCCACC
GCCTATATGGAGCTCAGTGACCTCCGGTCCGAGGATACCGCTGTGTATTT
CTGCGCCAGAGGTGGGAGCTGGCATTATTATTTTTACTACGGTCTCGACG
TCTGGGGCCAGGGCACTACCGTGACTGTGTCTTCCGCTAGCACCAAGGGC
CCATCCGTCTTCCCCCTGGCACCCTCCTCCAAGAGCACCTCTGGGGGCAC
AGCGGCCCTGGGCTGCCTGGTCAAGGACTACTTCCCCGAACCGGTGACGG
TGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCC
CTCCAGCAGCTTGGGCACCCAGACCTACATCTGCAACGTGAATCACAAGC
CCAGCAACACCAAGGTGGACAAGAGAGTTGAGCCCAAATCTTGT
66G8
SEQ ID NO: 11
EVQLLESGGGLVQPGGSLRLSCAASGFTFSIYAMSWVRQAPGKGLEWVSA
ISGSGGGTFYADSVKGRFTISRDNSKNTLFLQMNSLRAEDTAVYYCAKDR
RIAVAGTFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSC
SEQ ID NO: 25
GAGGTGCAGCTCCTGGAGAGCGGCGGAGGGCTGG
TCCAACCCGGCGGCTCTCTGCGGCTGTCCTGTGCGGCTAGTGGATTTACC
TTCTCTATCTACGCTATGAGCTGGGTCCGTCAGGCACCGGGTAAGGGACT
CGAATGGGTGTCCGCTATCTCTGGCAGCGGCGGTGGCACTTTCTACGCCG
ACAGCGTTAAGGGTCGCTTCACCATCTCTCGTGACAACTCCAAGAATACC
CTGTTCCTCCAGATGAATTCCCTGCGCGCCGAGGACACTGCTGTTTATTA
CTGCGCGAAGGATCGGCGGATCGCCGTCGCTGGCACATTCGATTACTGGG
GCCAGGGTACTCTGGTGACCGTGTCCAGTGCTAGCACCAAGGGCCCATCC
GTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAG
CAGCTTGGGCACCCAGACCTACATCTGCAACGTGAATCACAAGCCCAGCA
ACACCAAGGTGGACAAGAGAGTTGAGCCCAAATCTTGT
66E8
SEQ ID NO: 12
EVQLLESGGGLVQPGGSLRLSCAASGFTFSSYAMSWVRQAPGKGLEWVSA
ISGSGAGTYYADSVKGRFTISRDNSKNTLYLQMNSLRAEDTAVYYCAKDR
VIAVAAVFDYWGQGTLVTVSSASTKGPSVFPLAPSSKSTSGGTAALGCLV
KDYFPEPVTVSWNSGALTSGVHTFPAVLQSSGLYSLSSWTVPSSSLGTQ
TYICNVNHKPSNTKVDKRVEPKSC
SEQ ID NO: 26
GAGGTGCAACTCCTGGAGTCCGGTGGAGGCCTGG
TGCAGCCCGGAGGATCTCTGAGACTGTCTTGCGCGGCCTCCGGATTCACT
TTCTCCTCCTACGCTATGTCTTGGGTGCGGCAGGCCCCCGGCAAGGGACT
CGAGTGGGTGTCCGCCATCTCCGGCTCCGGAGCCGGCACCTATTACGCGG
ACAGCGTGAAGGGCCGCTTCACCATCTCCCGCGACAACTCTAAGAACACT
CTGTACCTGCAGATGAACTCTCTGCGTGCAGAGGACACCGCTGTCTACTA
CTGCGCTAAGGATCGCGTGATTGCCGTCGCCGCTGTCTTCGACTACTGGG
GTCAGGGGACACTCGTGACCGTGTCCAGCGCTAGCACCAAGGGCCCATCC
GTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAG
CAGCTTGGGCACCCAGACCTACATCTGCAACGTGAATCACAAGCCCAGCA
ACACCAAGGTGGACAAGAGAGTTGAGCCCAAATCTTGT
66H5
SEQ ID NO: 13
QVQLVESGGGVVQPGRSLRLSCAASGFTFISYGMHWVRQAPGKGLEWVAV
IWFDGSINNYADSVKGRFTISRDNSKNMLYLQMNSLRAEDTALYYCTRAG
IVGASWFDPWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSC
SEQ ID NO: 27
CAGGTTCAGCTGGTGGAGAGTGGAGGTGGCGTCG
TCCAGCCAGGCCGCAGCCTGCGGCTCTCCTGTGCTGCTTCCGGCTTTACC
TTTATCTCTTACGGCATGCACTGGGTGCGCCAGGCCCCCGGCAAGGGGTT
GGAGTGGGTTGCTGTGATCTGGTTTGACGGCTCCATCAACAACTACGCCG
ATAGTGTGAAGGGACGCTTCACTATCAGCAGGGACAACAGCAAGAATATG
CTGTACCTGCAGATGAATTCCCTCCGCGCTGAAGACACCGCGCTGTACTA
CTGCACACGGGCTGGTATCGTGGGGGCTTCCTGGTTTGACCCATGGGGGC
AGGGTACTCTGGTGACTGTGTCCAGCGCTAGCACCAAGGGCCCATCCGTC
TTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGA
ACTCAGGCGCCCTGACCAGCGGCGTGCACACCTTCCCCGCTGTCCTACAG
TCCTCAGGACTCTACTCCCTCAGCAGCGTGGTGACCGTGCCCTCCAGCAG
CTTGGGCACCCAGACCTACATCTGCAACGTGAATCACAAGCCCAGCAACA
GCAAGGTGGACAAGAGAGTTGAGCCCAAATCTTGT
64H10
SEQ ID NO: 14
QVQLVESGGGWQPGRSLRLSCAASGETESYYYIHWVRQAPGKGLEWVAL
IWYDGSNKDYADSVKGRFTISRDNSKNTLYLHVNSLRAEDTAVYYCAREG
TTRRGFDYWGQGTLVTVSSASTKGPSVFPLAPSSKSTSGGTAALGCLVKD
YFPEPVTVSWNSGALTSGVHTFPAVLQSSGLYSLSSWTVPSSSLGTQTY
ICNVNHKPSNTKVDKRVEPKSC
SEQ ID NO: 28
CAGGTTCAGCTGGTCGAGAGCGGCGGCGGTGTCG
TGCAGCCCGGCCGCTCCCTCCGGCTGTCTTGTGCGGCCTCTGGGTTCACA
TTTAGCTACTATTACATCCACTGGGTGAGACAGGCTCCAGGTAAAGGACT
CGAGTGGGTGGCTCTGATCTGGTACGATGGGAGTAACAAAGACTACGCAG
ACAGTGTTAAAGGCAGATTCACCATTAGTCGCGATAATTCCAAGAATACC
CTGTACTTGCACGTCAACAGCCTGCGCGCCGAGGATACTGCTGTGTACTA
TTGCGCTCGCGAGGGCACTACAAGGAGAGGATTCGACTACTGGGGTCAGG
GCACCCTGGTCACAGTCAGCAGCGCTAGCACCAAGGGCCCATCCGTCTTC
CCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTT
GGGCACCCAGACCTACATCTGCAACGTGAATCACAAGCCCAGCAACACCA
AGGTGGACAAGAGAGTTGAGCCCAAATCTTGT
Olig 2125 (4970-45)
SEQ ID NO: 29
5′CACCATGGAGTTTGGGCTGAGCTG3′
Olig 2122 (4970-41)
SEQ ID NO: 30
5′CACCATGGACTGGACCTGGAGGG3′
Anti-sense (AS)(Olig 4001) (5590-33)
SEQ ID NO: 31
5′CATTATGCACCTCCACGCCGTCCAC3′

What is claimed is:

1. A bispecific antigen binding protein, comprising:
a) a first polypeptide comprising a first heavy chain variable region (VH1), wherein the VH1 is fused through its C-terminus to the N-terminus of a CH1 domain and wherein the VH1 comprises three CDRs and binds to a first epitope, and
b) a second polypeptide comprising a second heavy chain variable region (VH2), wherein the VH2 is fused through its C-terminus to the N-terminus of a CL domain and wherein the VH2 comprises three CDRs and binds to a second epitope.

2. The antigen binding protein accordingly to claim 1, wherein the first and second epitopes are located on the same antigen.

3. The antigen binding protein accordingly to claim 1, wherein the first and second epitopes are located on different antigens.

4. The antigen binding protein according to claim 1, wherein
i) the VH1 or CH1 domain of the first polypeptide comprises at least one amino acid substitution to introduce a charged amino acid; and
ii) the VH2 or CL domain of the second polypeptide comprises at least one amino acid substitution to introduce a charged amino acid with a charge opposite of the substituted amino acid of the first polypeptide.

5. The antigen binding protein according to claim 4, wherein
i) the CH1 domain of the first polypeptide comprises at least one amino acid substitution to introduce a negatively charged amino acid; and
ii) the CL domain of the second polypeptide comprises at least one amino acid substitution to introduce a positively charged amino acid.

6. The antigen binding protein according to claim 4, wherein
i) the CH1 domain of the first polypeptide comprises at least one amino acid substitution to introduce a positively charged amino acid; and
ii) the CL domain of the second polypeptide comprises at least one amino acid substitution to introduce a negatively charged amino acid.

7. The antigen binding protein according to claim 4, wherein the amino acid substitution in the CH1 domain of the first polypeptide corresponds to position 183 using EU numbering, and the amino acid substitution in the CL domain of the second polypeptide corresponds to position 176 using EU numbering.

8. The antigen binding protein according to claim 7, wherein the amino acid substitution in the CH1 domain of the first polypeptide corresponds to S183E using EU numbering, and the amino acid substitution in the CL domain of the second polypeptide corresponds to S176K using EU numbering.

9. The antigen binding protein according to claim 7, wherein the amino acid substitution in the CH1 domain of the first polypeptide corresponds to S183K using EU numbering, and the amino acid substitution in the CL domain of the second polypeptide corresponds to S176E using EU numbering.

10. The antigen binding protein according to any preceding claim, wherein the first polypeptide chain is an antibody heavy chain.

11. The antigen binding protein according to claim 10, wherein the antigen binding protein comprises two first polypeptides and two second polypeptides.

12. A method of agonizing a receptor comprising contacting the receptor with a bispecific receptor binding protein, wherein the bispecific receptor binding protein comprises:
a) a first polypeptide comprising a first heavy chain variable region (VH1), wherein the VH1 is fused through its C-terminus to the N-terminus of a CH1 domain and wherein the VH1 comprises three CDRs and binds to a first epitope, and
b) a second polypeptide comprising a second heavy chain variable region (VH2), wherein the VH2 is fused through its C-terminus to the N-terminus of a CL domain and wherein the VH2 comprises three CDRs and binds to a second epitope,
wherein the first and second epitopes are both located on the receptor.

13. The method according to claim 12, wherein
i) the VH1 or CH1 domain of the first polypeptide comprises at least one amino acid substitution to introduce a charged amino acid; and
ii) the VH2 or CL domain of the second polypeptide comprises at least one amino acid substitution to introduce a charged amino acid with a charge opposite of the substituted amino acid of the first polypeptide.

14. The method according to claim 13, wherein
i) the CH1 domain of the first polypeptide comprises at least one amino acid substitution to introduce a negatively charged amino acid; and
ii) the CL domain of the second polypeptide comprises at least one amino acid substitution to introduce a positively charged amino acid.

15. The method according to claim 13, wherein
i) the CH1 domain of the first polypeptide comprises at least one amino acid substitution to introduce a positively charged amino acid; and
ii) the CL domain of the second polypeptide comprises at least one amino acid substitution to introduce a negatively charged amino acid.

16. The method according to claim 13, wherein the amino acid substitution in the CH1 domain of the first polypeptide corresponds to position 183, and the amino acid substitution in the CL domain of the second polypeptide corresponds to position 176.

17. The method according to claim 16, wherein the amino acid substitution in the CH1 domain of the first polypeptide corresponds to S183E using EU numbering, and the amino acid substitution in the CL domain of the second polypeptide corresponds to S176K using EU numbering.

18. The method according to claim 16, wherein the amino acid substitution in the CH1 domain of the first polypeptide corresponds to S183K using EU numbering, and the amino acid substitution in the CL domain of the second polypeptide corresponds to S176E using EU numbering.

19. The method according to claim according to any of claims 12-18, wherein the first polypeptide chain is an antibody heavy chain.

20. The antigen binding protein according to claim 19, wherein the antigen binding protein comprises two first polypeptides and two second polypeptides.

21. A tetra-specific, tetravalent antigen binding protein, comprising:
a) a first antibody heavy chain comprising a first heavy chain variable region (VH1), wherein the VH1 is fused through its C-terminus to the N-terminus of the CH1 domain of the first antibody heavy chain and wherein the VH1 comprises three CDRs and binds to a first epitope;
b) a first polypeptide comprising a second heavy chain variable region (VH2), wherein the VH2 is fused through its C-terminus to the N-terminus of a CL domain and wherein the VH2 comprises three CDRs and binds to a second epitope;
c) a second antibody heavy chain comprising a third heavy chain variable region (VH3), wherein the VH3 is fused through its C-terminus to the N-terminus of the CH1 domain of a second antibody heavy chain and wherein the VH3 comprises three CDRs and binds to a third epitope; and
d) a second polypeptide comprising a fourth heavy chain variable region (VH4), wherein the VH4 is fused through its C-terminus to the N-terminus of a CL domain and wherein the VH4 comprises three CDRs and binds to a second epitope.

22. The antigen binding protein accordingly to claim 21, wherein the first and second epitopes are located on a first antigen and the third and fourth epitopes are located on a second antigen.

23. The antigen binding protein accordingly to claim 21, wherein the first, second, third, and fourth epitopes are located on different antigens.

24. The antigen binding protein according to claim 21, wherein
i) the CH3 domain of the first antibody heavy chain comprises at least one amino acid substitution to introduce a charged amino acid; and
ii) the CH3 domain of the second heavy chain comprises at least one amino acid substitution to introduce a charged amino acid with a charge opposite of the substituted amino acid of the CH3 domain of the first heavy chain.

25. The antigen binding protein according to claim 24, wherein
i) the CH3 domain of the first heavy chain comprises at least two amino acid substitutions to introduce amino acids of the same charge; and
ii) the CH3 domain of the second heavy chain comprises at least two amino acid substitutions to introduce amino acids both with a charge opposite of the substituted amino acids of the CH3 domain of the first heavy chain.

26. The antigen binding protein according to claim 25, wherein
i) the CH3 domain of the first heavy chain comprises at least two amino acid substitutions to introduce two negatively charged amino acids; and
ii) the CH3 domain of the second heavy chain comprises at least two amino acid substitutions to introduce two positively charged amino acids.

27. The antigen binding protein according to claim 25, wherein
i) the CH3 domain of the first heavy chain comprises at least two amino acid substitutions to introduce two positively charged amino acids; and
ii) the CH3 domain of the second heavy chain comprises at least two amino acid substitutions to introduce two negatively charged amino acids.

28. The antigen binding protein according to claim 24, wherein
i) the CH3 domain of the first heavy chain comprises at least one amino acid substitution at a position selected from the group consisting of residues corresponding to positions 356, 399, and 357 using EU numbering; and
ii) the CH3 domain of the second heavy chain comprises at least one amino acid substitution at a position selected from the group consisting of residues corresponding to positions 392, 409, and 370 using EU numbering.

29. The antigen binding protein according to claim 24, wherein
i) the CH3 domain of the first heavy chain comprises at least one amino acid substitution at a position selected from the group consisting of residues corresponding to positions 392, 409, and 370 using EU numbering; and
ii) the CH3 domain of the second heavy chain comprises at least one amino acid substitution at a position selected from the group consisting of residues corresponding to positions 356, 399, and 357 using EU numbering.

30. The antigen binding protein according to claim 28, wherein
i) the CH3 domain of the first heavy chain comprises at least two amino acid substitutions at at least two positions selected from the group consisting of residues corresponding to positions 356, 399, and 357 using EU numbering; and
ii) the CH3 domain of the second heavy chain comprises at least two amino acid substitutions at at least two positions selected from the group consisting of residues corresponding to positions 392, 409, and 370 using EU numbering.

31. The antigen binding protein according to claim 29, wherein
i) the CH3 domain of the first heavy chain comprises at least two amino acid substitutions at at least two positions selected from the group consisting of residues corresponding to positions 392, 409, and 370 using EU numbering; and
ii) the CH3 domain of the second heavy chain comprises at least two amino acid substitutions at at least two positions selected from the group consisting of residues corresponding to positions 356, 399, and 357 using EU numbering.

32. The antigen binding protein according to claim 30, wherein
i) the CH3 domain of the first heavy chain comprises at least two amino acid substitutions selected from the group consisting of residues corresponding to E356K, D399K, and E357K using EU numbering; and
ii) the CH3 domain of the second heavy chain comprises at least two amino acid substitutions at at least two positions selected from the group consisting of residues corresponding to K392D, K409D, and K370D using EU numbering.

33. The antigen binding protein according to claim 31, wherein
i) the CH3 domain of the first heavy chain comprises at least two amino acid substitutions selected from the group consisting of residues corresponding to K392D, K409D, and K370D using EU numbering; and
ii) the CH3 domain of the second heavy chain comprises at least two amino acid substitutions at at least two positions selected from the group consisting of residues corresponding to E356K, D399K, and E357K using EU numbering.

34. The antigen binding protein according to claim 32, wherein
i) the CH3 domain of the first heavy chain comprises at least two amino acid substitutions of residues corresponding to K392D and K409D using EU numbering; and
ii) the CH3 domain of the second heavy chain comprises at least two amino acid substitutions of residues corresponding to E356K and D399K using EU numbering.

35. The antigen binding protein according to claim 33, wherein
i) the CH3 domain of the first heavy chain comprises at least two amino acid substitutions of residues corresponding to E356K and D399K using EU numbering; and
ii) the CH3 domain of the second heavy chain comprises at least two amino acid substitutions of residues corresponding to K392D and K409D using EU numbering.

36. The antigen binding protein according to claim 21, wherein
i) the VH1 or CH1 domain of the first heavy chain comprises at least one amino acid substitution to introduce a charged amino acid;
ii) the VH2 or CL domain of the first polypeptide comprises at least one amino acid substitution to introduce a charged amino acid with a charge opposite of the substituted amino acid of the first heavy chain;
iii) the VH1 or CH1 domain of the second heavy chain polypeptide comprises at least one amino acid substitution to introduce a charged amino acid with a charge opposite of the substituted amino acid of the first heavy chain; and
iv) the VH2 or CL domain of the second polypeptide comprises at least one amino acid substitution to introduce a charged amino acid with a charge opposite of the substituted amino acid of the second heavy chain.

37. The antigen binding protein according to claim 36, wherein
i) the CH1 domain of the first heavy chain comprises at least one amino acid substitution to introduce a negatively charged amino acid;
ii) the CL domain of the first polypeptide comprises at least one amino acid substitution to introduce a positively charged amino acid;
iii) the CH1 domain of the second heavy chain polypeptide comprises at least one amino acid substitution to introduce a positively charged amino acid; and
iv) the CL domain of the second polypeptide comprises at least one amino acid substitution to introduce a negatively charged amino acid.

38. The antigen binding protein according to claim 36, wherein
i) the CH1 domain of the first heavy chain comprises at least one amino acid substitution to introduce a positively charged amino acid;
ii) the CL domain of the first polypeptide comprises at least one amino acid substitution to introduce a negatively charged amino acid;
iii) the CH1 domain of the second heavy chain polypeptide comprises at least one amino acid substitution to introduce a negatively charged amino acid; and
iv) the CL domain of the second polypeptide comprises at least one amino acid substitution to introduce a positively charged amino acid.

39. The antigen binding protein according to claim 37, wherein the amino acid substitution in the CH1 domain of the first heavy chain corresponds to S183E using EU numbering, the amino acid substitution in the CL domain of the first polypeptide corresponds to S176K using EU numbering; the amino acid substitution in the CH1 domain of the second heavy chain corresponds to S183K using EU numbering, and the amino acid substitution in the CL domain of the second polypeptide corresponds to S176E using EU numbering.

40. The antigen binding protein according to claim 38, wherein the amino acid substitution in the CH1 domain of the first heavy chain corresponds to S183K using EU numbering, the amino acid substitution in the CL domain of the first polypeptide corresponds to S176E using EU numbering; the amino acid substitution in the CH1 domain of the second heavy chain corresponds to S183E using EU numbering, and the amino acid substitution in the CL domain of the second polypeptide corresponds to S176K using EU numbering.
